(19) United States
(12) Patent Application Publication
(10) Pub. No.: US 2022/0403371 A1
(43) Pub. Date: Dec. 22, 2022

(54) CHROMOSOME CONFORMATION CAPTURE FROM TISSUE SAMPLES

(71) Applicant: PHASE GENOMICS, INC., Seattle, WA (US)

(72) Inventors: Shawn SULLIVAN, Seattle, WA (US); Emily REISTER MORRIS, Seattle, WA (US); Kyle LANGFORD, Seattle, WA (US); Ivan LIACHKO, Seattle, WA (US); Stephen M. EACKER, Seattle, WA (US)

(21) Appl. No.: 17/774,756

(22) PCT Filed: Nov. 13, 2020
(86) PCT No.: PCT/US2020/060511

§ 371 Date: May 5, 2022

Related U.S. Application Data
(60) Provisional application No. 62/936,042, filed on Nov. 15, 2019.

(51) Int. Cl. C12N 15/10; C12Q 1/6806

(52) U.S. Cl. CPC C12N 15/1006; C12Q 1/6806

(57) ABSTRACT

Provided herein are methods and systems for identifying chromosomal structural variants in a preserved sample obtained from a subject using focused acoustic energy and chromosomal conformational capture. Also provided herein are methods and systems for relating the chromosomal structural variants identified from the preserved tissue sample to diseases or disorders, and methods of treating same.

### RELATED APPLICATIONS

This application claims the benefit of priority to U.S. Provisional Application No. 62/936,042 filed Nov. 15, 2019, which is hereby incorporated by reference in its entirety for all purposes.

### BACKGROUND

The detection of chromosomal abnormalities is a frontline diagnostic for a variety of hematological cancers. Even state-of-the-art cancer cytogenetic methods have limitations that often require the use of multiple tests for diagnosis. Karyotyping methods offer a genome-wide view of chromosomal aberrations but have limited resolution. Methods like fluorescence in situ hybridization (FISH) allow only one or in some cases a few loci to be interrogated at a time. Chromosomal microarray analysis (CMA) is unable to call balanced translocations, inversions, elucidate complex rearrangements, and changes in ploidy. Furthermore, from a cancer diagnostic purposes, CMA is somewhat limited by the percent tumor composition of a sample, with an operational sensitivity in 20% abundance range. And while CMA and FISH can be applied to solid tumors in some cases, karyotyping is not a method that can be routinely applied to solid tumors. As such, the utility of cytogenomic methods in solid tumor biomarker discovery has lagged. There thus exists a need in the art for additional methods that accurately and rapidly identify chromosomal structural variants.

The present invention would address these needs by providing methods that accurately and rapidly identify chromosomal structural variants using chromosomal conformational capture methods.

### SUMMARY

In one aspect, provided herein is a method comprising: providing a tissue sample in a solution in a vessel, the tissue sample comprising nucleic acid material; dissociating the tissue sample by exposing the tissue sample and the solution in the vessel to focused acoustic energy to release the nucleic acid material from the tissue sample; recovering the nucleic acid material; and performing chromosome conformation capture analysis on the nucleic acid material. In some cases, the solution is a non-solvent solution. In some cases, the tissue sample is a preserved tissue sample. In some cases, the tissue sample is a cross-linked tissue sample. In some cases, the tissue sample is a formalin fixed paraffin-embedded (FFPE) sample. In some cases, the disassociating step comprises exposing the FFPE sample to focused acoustic energy for a time sufficient to disassociate enough paraffin from the FFPE sample to allow recovery of the nucleic acid material from the tissue sample. In some cases, the disassociating step comprises disassociating more than 90% of paraffin attached to the FFPE sample. In some cases, the disassociating step comprises disassociating more than 98% of paraffin attached to the FFPE sample. In some cases, the disassociating step comprises rehydrating the tissue sample while exposing the tissue sample to focused acoustic energy. In some cases, the disassociating step comprises maintaining a temperature of the solution at about 5° C. to about 60° C. or about 18° C. to about 20° C. In some cases, the tissue sample has a thickness of 5 to 25 microns and a length of less than 25 mm. In some cases, the dissociating step comprises adding a protease to the solution and the tissue sample in the vessel prior to exposing the tissue sample to focused acoustic energy. In some cases, comprising inactivating the protease. In some cases, the inactivating the protease comprises heating the vessel to about 98° C. In some cases, the method comprises maintaining the tissue sample in the vessel at below 50° C. until heating with sample to 90-100° C. In some cases, the focused acoustic energy has a duty factor of between 10% and 30%. In some cases, the focused acoustic energy has a duty factor of about 15% or about 20%. In some cases, the focused acoustic energy has a peak intensity power of between 60W and 90W. In some cases, the focused acoustic energy has a peak intensity power of about 75W. In some cases, the method further comprises performing a second dissociating step comprising exposing the tissue sample and the solution in the vessel to focused acoustic energy to release additional nucleic acid material from the tissue sample while maintaining the vessel at about 4° C. to about 7° C. In some cases, the focused acoustic energy has a duty factor of between 10% and 30%. In some cases, the focused acoustic energy has a duty factor of about 15% or about 20%. In some cases, the focused acoustic energy has a peak intensity power of between 60W and 90W. In some cases, the focused acoustic energy has a peak intensity power of about 75W. In some cases, the method further comprises isolating supernatant following the dissociating step in a vessel, adding additional solution to the vessel comprising the tissue sample and performing a second dissociating step on the tissue sample comprising exposing the tissue sample and the additional solution in the vessel to focused acoustic energy to release additional nucleic acid material from the tissue sample while maintaining the vessel at about 5° C. to about 60° C. or about 18° C. to about 20° C. In some cases, the focused acoustic energy has a duty factor of between 10% and 30%. In some cases, the focused acoustic energy has a duty factor of about 15% or about 20%. In some cases, the focused acoustic energy has a peak intensity power of between 60W and 90W. In some cases, the focused acoustic energy has a peak intensity power of about 75W. In some cases, the method further comprises isolating supernatant following the second dissociating step in a vessel, performing a third dissociating step on both the supernatant isolated following the second dissociating step and the supernatant isolated prior to the second dissociating step by exposing each of the supernatants to focused acoustic energy while maintaining the temperature of the vessels comprising the supernatants at about 4° C. to about 7° C. and combining the supernatants. In some cases, the focused acoustic energy has a duty factor of between 10% and 30%. In some cases, the focused acoustic energy has a duty factor of about 15% or about 20%. In some cases, the focused acoustic energy has a peak intensity power of between 60W and 90W. In some cases, the focused acoustic energy has a peak intensity power of about 75W. In some cases, the dissociating step comprises exposing the tissue sample to focused acoustic energy at an intensity suitable to avoid shearing the nucleic acid material. In some cases, a majority of the fragments of nucleic acid material after exposing the tissue sample to focused acoustic energy have a size of 1000 bp or greater. In some cases, the dissociating step preserves formaldehyde crosslinks in the tissue sample. In some cases, the focused acoustic energy has a frequency of between about 100 kilohertz and about 100 megahertz; the focused acoustic energy has a focal zone with a width of less than about 2 centimeters; and/or the focused acoustic energy originates from an acoustic energy source spaced from and exterior to the vessel, wherein at least a portion of the acoustic energy propagates exterior to the vessel. In some cases, the recovering step comprises centrifuging the tissue sample, thereby separating a supernatant solution containing nucleic acid material dissociated from insoluble contaminants. In some cases, the recovering step comprises purifying nucleic acid material by solid phase reversible immobilization. In some cases, performing chromosome conformation capture analysis on the nucleic acid material comprises: proximity ligating the nucleic acid material to form a library of proximity-ligated polynucleotides and identifying paired polynucleotide sequences in the library of proximity-ligated polynucleotides. In some cases, performing chromosome conformation capture analysis on the nucleic acid material comprises: fragmenting the nucleic acid material, proximity ligating the nucleic acid material to form a library of proximity-ligated polynucleotides, and identifying paired polynucleotide sequences in the library of proximity-ligated polynucleotides. In some cases, the identifying step comprising sequencing the proximity ligations.

### BRIEF DESCRIPTION OF THE DRAWINGS

FIGS. 1A-1E show an overview of an illustrative proximity ligation method to detect cytogenomic aberrations. (FIG. 1A) Cells from an individual are cross-linked, forming covalent bonds between chromatin in close proximity in the intact nucleus. (FIG. 1B) Frequency interactions captured by Hi-C are related to the proximity of the two sequences based on the linear distance between them on a chromosome. (FIG. 1C) A HiC interaction matrix from a karyotypically normal cell line. (FIG. 1D) A HiC matrix from a cell line containing a translocation between chr4 and chr11 observed by off-diagonal signal on the heat map (dashed gray box) and observed very clearly at a higher zoom of the region (FIG. 1E).

FIG. 2 shows HiC-QC computed statistics for HiC libraries generated from Phase Genomics FFPE Hi-C methods.

FIGS. 3A-3D show analysis of clinical samples by HiC methods provided throughout this disclosure (FIG. 3A). All clinical samples exceed HiC-QC-measured quality standard. (FIG. 3B) Sample translocation and (FIG. 3C) deletion or amplifications observed in clinical Hi-C data. (FIG. 3D) Summary of detected aberrations that overlap with combined karyotype, FISH, and CMA data available for clinical samples. Only aberrations detectable at 20% abundance (limit of CMA detection) were considered.

FIG. 4 shows an outline of Hi-C methodology. DNA sequences in close physical proximity are cross-linked during formalin fixation, fragmented by restriction digest and ligated together. Sequencing adapters are added and chimeric molecules are sequenced. Mapping reads 1 and 2 relative to each other creates a contact matrix heat which allows identification of chromosomal rearrangements.

FIG. 5A-5B shows the utility of AFA methods to generate Hi-C libraries on clinical samples. Libraries generated using above described methods from a single section of FFPE breast (FIG. 5A) or ovary (FIG. 5B) tumor sample is sufficient to identify non-reciprocal translocations between chromosomes X and 8 (FIG. 5A) and chromosomes 4 and 7 (FIG. 5B).

### DETAILED DESCRIPTION

Provided herein are methods and systems for the identification of chromosomal structural variants using chromatin conformation capture techniques. In some embodiments, the disclosure further provides systems and methods for detecting chromosomal structural variants in tissue samples previously known to be refractory to karyotyping or karyotyping by sequencing (KBS) analyses (e.g., solid tissue or tumor samples). In some embodiments, the disclosure further provides systems and methods for relating chromosomal structural variants to biological information pertinent to the chromosomal structural variant (for example, clinical data). The chromatin conformation capture (3-C) techniques and systems and methods for relating chromosomal structural variants to biological information pertinent to specific chromosomal structural variants for use in the methods and systems provided herein can be those CCC techniques, systems and methods described in WO 2020/198704, which is incorporated herein by reference in its entirety.

In one embodiment, a method for identifying chromosomal structural variants provided herein comprises: (a) providing a tissue sample in a solution in a vessel, the tissue sample comprising nucleic acid material; (b) dissociating the tissue sample by exposing the tissue sample and the solution in the vessel to focused acoustic energy to release the nucleic acid material from the tissue sample; (c) recovering the nucleic acid material; and (d) performing chromosome conformation capture analysis on the nucleic acid material. The tissue sample can be a solid tumor sample. The tissue sample (e.g., solid tumor sample) can be a preserved tissue sample. The tissue sample (e.g., solid tumor sample) can be paraffin-embedded. The tissue sample (e.g., solid tumor sample) can be cross-linked or fixed. In one embodiment, the tissue sample is a formalin fixed paraffin-embedded (FFPE) sample. The dissociating of step (b) can be repeated one or more times. In one embodiment, the dissociating of step (b) is repeated once on the tissue sample and the solution in the vessel. In another embodiment, the method further comprises: (i) isolating the solution in the vessel following step (b) and prior to step (c); (ii) adding an additional volume of solution to the tissue sample remaining in the vessel from step (i); (iii) repeating the dissociating of step (b) on the tissue sample in the vessel to which the additional volume of solution was added; (iv) isolating the additional volume of solution added to the tissue sample in the vessel following the additional dissociating step; (v) dissociating the solutions isolated in steps (i) and (iv) by exposing said solutions to focused acoustic energy to release additional nucleic acid material from any remaining portions of the tissue sample in said solutions; and (vi) combining the solutions subjected to step (v). In one embodiment, the method further comprises repeating steps (i)-(v) one or more times. The solution used in each dissociating step can be a non-solvent solution. The non-solvent solution can be any solution that does not contain a solvent that can cause damage to the nucleic acid and/or proteinaceous material contained within the tissue sample exposed to any of the methods provided herein. The non-solvent solution can include water and a detergent.

Chromatin conformation capture methods, such as 3-C, 4-C, 5-C, and Hi-C, physically link DNA molecules in close proximity inside intact cells. These methods measure how often two loci co-associate in space in vivo. A two-dimensional contact matrix is then calculated from chromatin conformation capture data by mapping high throughput sequencing reads from a chromatin conformation capture library to a draft or reference genome. In a contact matrix, loci originating from the same chromosomes have a higher interaction frequency than loci on different chromosomes, and neighboring loci on the same chromosome have a higher interaction frequency than distal loci on that chromosome. Every individual's genome exhibits a slightly different contact matrix due to allelic variation within the individual's population of cells and mutations the individual was born with or acquired during their lifetime. These differences are termed variants. Some variants can be seen with the naked eye by visualizing the contact matrix as a contact map. Other variants can be detected by analyzing the contact matrix computationally. These variants include, but are not limited to, balanced and unbalanced translocations, inversions, and copy number variation such as insertions, deletions, repeat expansions, and other complex events. Some variants are known to have clinical significance, i.e. are associated with a disease and/or course of treatment. Other variants are of unknown clinical significance, or are novel (not previously described in the art). Chromatin conformation data and the methods and systems disclosed herein provide the means to describe variants of known clinical significance, and to discover variants of unknown clinical significance and novel variants.

Karyotyping by sequencing (KBS) methods of the disclosure use chromatin conformation data in clinical and research scenarios utilizing solid tissue samples (e.g., solid tumors) where karyotyping or karyotype-like data would be useful. This method includes multiple major applications. First, KBS methods are able to identify human genomic rearrangements observable by cytogenetic methods and to test for the presence of known clinically-reportable variants, in effect producing the same kind of actionable information as karyotyping but with highly different, powerful means. Second, KBS methods are capable of analyzing any sample to detect any structural variants, and classify these variants using any provided data about structural variation in the organism being sampled.

### Subjects

The disclosure provides methods and systems for detecting one or more chromosomal structural variants in a sample obtained from a subject. The samples can include biopsy samples, surgical samples, tumor samples, whole organs, and other samples.

The subject can be any organism. In some embodiments, the subject is a eukaryote. In some embodiments, the subject is a metazoan. In some embodiments, the subject is a vertebrate. In some embodiments, the subject is a mammal. In some embodiments, the subject is a human, a monkey, an ape, a rabbit, a guinea pig, a gerbil, a rat or a mouse. In some embodiments, the subject is an agricultural animal. Exemplary agricultural animals include horses, sheep, cows, pigs and chickens. In some embodiments, the subject is an animal that is kept as a pet (a veterinary subject). Exemplary pets include dogs and cats.

In some embodiments, the subject is a human.

In some embodiments, particularly those embodiments wherein the subject is a human, the subject has one or more symptoms of a disease or disorder which is caused by one or more chromosomal structural variants in the subject. In some embodiments, the chromosomal structural variant is one that is known in the art to cause a disease or disorder, to affect the function of a gene or genes that cause a disease or disorder. The disease or disorder can be any disease or disorder known in the art and/or provided herein to be associated with or caused by one or more chromosomal structural variants. In alternative embodiments, the chromosomal structural variant is a novel chromosomal structural variant, i.e. a variant that has not previously been described in the art. The disclosure provides systems and methods to identify both novel and known chromosomal structural variants.

The disclosure provides methods and systems for detecting one or more chromosomal structural variants in tissues and/or cells isolated or derived from any tissue or cell type in the subject. In some embodiments, the tissue is a healthy tissue of the subject, for example, healthy skin, bone marrow, liver, kidney, neural tissue or muscle. In some embodiments, the tissue has one or more symptoms of a disease or disorder. In some embodiments, the disease or disorder is cancer, and the tissue comprises cancer cells. In some embodiments, the cancer comprises a solid tumor and the tissue comprises tumor cells. In some embodiments, the tissue comprises a mixture of cells that comprise one or more chromosomal structural variants and cells that do not comprise one or more chromosomal structural variants. The tissue can be fresh. The tissue can be fresh-frozen. The tissue can be fixed. The tissue can be preserved. In one embodiment, the tissue is paraffin-embedded. In another embodiment, the tissue is formalin-fixed and paraffin-embedded (FFPE). In some cases, the tissue sample has a thickness of 5 to 25 microns and a length of less than 25 mm. In some cases, the tissue samples are curls (sections that are 10 microns or greater). The curls can be FFPE curls.

In one embodiment, a sample (e.g., a biopsy) is taken from a patient and placed in a fixative (e.g., formalin) during a medical procedure. This fixed sample can be subsequently analyzed using the techniques of the present disclosure. For example, genomic features such as rearrangements relevant to cancer can be identified.

In one embodiment, provided herein are methods and systems for detecting one or more chromosomal structural variants in preserved samples from any tissue or cell type in the subject. The samples can be stored pursuant to basic research, translation research, a surgical excision or archived pursuant to a drug trial. The preserved sample can be cross-linked for example using at least one of a formaldehyde, a formalin, UV light, mitomycin C, nitrogen mustard, melphalan, 1,3-butadiene diepoxide, cis diaminedichloroplatinum(II) and cyclophosphamide. Alternatively, the preserved sample can be cross-linked using formalin. The preserved sample can maintain positional information as to nucleic acids within it. In one embodiment, the preserved sample is an embedded sample such as a formalin fixed paraffin-embedded (FFPE) sample. The preserved samples can be fixed directly and without homogenization, in some cases, by dropping the sample into a fixative solution.

In one embodiment, the preserved tissue sample is treated to isolate nucleic acids such that protein DNA complexes are not destroyed. In some cases, the protein DNA complexes are isolated such that a first nucleic acid segment and a second nucleic acid segment in close proximity are held together independent of a phosphodiester backbone. In some cases, the preserved tissue sample is treated by protecting the sample from boiling conditions. In some cases, the preserved tissue sample is treated at a temperature not greater than 40° C. In one embodiment, the DNA protein complexes comprise chromatin. In some cases, the preserved tissue sample preserves positional information reflective of its configuration in a tissue. In one embodiment, the preserved tissue sample is not homogenized during preservation or prior to isolating nucleic acids, such that positional information of a DNA protein complex excised from the sample is preserved and available as part of the genome structural analysis.

The preserved tissue sample can be stored for at least 1 day, 2 days, 3 days, 4 days, 5 days, 6 days, 7 days, 8, days, 9 days, 10 days, 11 days, 12 days, 13 days, 2 weeks, 3 week, 1 month, 1.5 months, 2 months, 2.5 months, 3 months, 3.5 month, 4 months, 4.5 months, 5 months, 5.5 months, 6 months, 8 months, 10 months, 1 year, 2 years, 3 years, 4, years, 5 years, 10 years, 15 years, 20 years, 25 years, 30 years, 35 years, 40 years, 45 years, or 50 years. The preserved tissue sample can be stored for at most 1 day, 2 days, 3 days, 4 days, 5 days, 6 days, 7 days, 8, days, 9 days, 10 days, 11 days, 12 days, 13 days, 2 weeks, 3 week, 1 month, 1.5 months, 2 months, 2.5 months, 3 months, 3.5 month, 4 months, 4.5 months, 5 months, 5.5 months, 6 months, 8 months, 10 months, 1 year, 2 years, 3 years, 4, years, 5 years, 10 years, 15 years, 20 years, 25 years, 30 years, 35 years, 40 years, 45 years, or 50 years. The preserved tissue sample can be stored for about 1 day, 2 days, 3 days, 4 days, 5 days, 6 days, 7 days, 8, days, 9 days, 10 days, 11 days, 12 days, 13 days, 2 weeks, 3 week, 1 month, 1.5 months, 2 months, 2.5 months, 3 months, 3.5 month, 4 months, 4.5 months, 5 months, 5.5 months, 6 months, 8 months, 10 months, 1 year, 2 years, 3 years, 4, years, 5 years, 10 years, 15 years, 20 years, 25 years, 30 years, 35 years, 40 years, 45 years, or 50 years. In one embodiment, the preserved tissue sample is stored for at least one week prior to isolating nucleic acids. In one embodiment, the preserved tissue sample is stored for at least 6 months prior to isolating nucleic acids.

The preserved tissue sample can be transported from a collection point prior to isolating nucleic acids. The preserved tissue sample can be collected in a sterile environment. The preserved tissue sample can be positioned in a nonsterile environment prior to isolating nucleic acids.

Preserved samples, such as formalin-fixed, paraffin embedded samples, often comprise nucleic acids having damage, such as damage caused by fixative and/or embedding materials. A relevant component in making use of DNA is preserving the integrity of DNA physical linkage information of isolated DNA subject to a DNA damaging agent. Although DNA is a relatively stable molecule, the integrity of DNA can be subject to environmental factors and particularly time. The presence of nuclease contamination, hydrolysis, oxidation, chemical, physical and mechanical damages represent some of the major threats to DNA preservation. The mechanical, environmental and physical factors encountered by DNA during transportation frequently leave them in fragments and potentially lose long-range information, which are critical for genomic analysis. Existing methods for preserving DNA information mostly delay the decay of DNA but provide little protection to DNA damage over time, especially when fragmentation occurs. In many cases, such DNA damage can be mitigated by fixing and embedding samples intended for long term storage. For example, FFPE (formalin-fixation, paraffin embedded) samples can be preserved for a long time. However, the preservation process can result in DNA damage. Additionally, later DNA extraction methods can often be harsh and lead to further DNA damage and fragmentation.

Disclosed herein are methods and systems related to recovering long-distance genomic information from preserved and/or stored nucleic acid molecules, such as nucleic acid molecules in DNA complexes or chromatin aggregates, such as cross-linked chromatin stored in preserved (e.g., FFPE) samples (including tissue-based preserved samples and cell culture-based preserved samples). Methods and systems provided herein can be used for the recovery of nucleic acid samples from these preserved samples such that nucleic acid physical linkage information is preserved. Physical linkage information is preserved either by preservation of the nucleic acids themselves in the FFPE extraction process, or by preserving nucleic acid complexes such that physical linkage information is preserved independent of any damage that may occur to the nucleic acids themselves in the extraction process.

### Adaptive Focused Acoustics (AFA)-Based Nucleic Acid Extraction

In one embodiment, provided herein are methods and systems for detecting one or more chromosomal structural variants in nucleic acid obtained, derived or extracted from preserved samples from any tissue or cell type in the subject using focused acoustic energy. In one embodiment, isolation or extraction of nucleic acid from a preserved sample (e.g., FFPE tissue sample) utilizes focused acoustic energy and an acoustic treatment device as described in WO2014078650, which is herein incorporated by reference and described briefly below.

In one embodiment the preserved sample is an FFPE sample (e.g., solid tumor FFPE sample) and the paraffin is disassociated from the FFPE sample using a non-solvent solution. In one embodiment, the non-solvent solution does not contain or expose the FFPE sample to a solvent during the process of paraffin disassociation. The non-solvent solution can include water and/or a detergent. The non-solvent solution may be used together with suitable focused acoustic energy to disassociate paraffin from the FFPE sample. Such paraffin disassociation may be done without exposing the sample to relatively high temperatures. For example, the paraffin may be suitably disassociated from the sample while maintaining the sample temperature below 5-60° C. The paraffin may be suitably dissociated from the sample while maintaining the sample temperature between 1-30° C. The paraffin may be suitably dissociated from the sample while maintaining the sample temperature from about 18-20° C. or from about 4-7° C. In one embodiment, the sample temperature is maintained at, approximately 20° C. In another embodiment, the sample temperature is maintained at approximately 7° C.). The paraffin disassociation utilized herein can increase nucleic acid material yield by at least 2 to 4 times than found with processes known in the art for extraction nucleic acid from FFPE. In one embodiment, paraffin disassociation using the focus acoustic energy method described herein occurs in 3 minutes or less.

In one embodiment, the sample is rehydrated during the paraffin disassociation process. Rehydration can serve to improve bio-material yield as well.

In one embodiment, the preserved tissue for use in the methods and systems provided herein is an FFPE sample and the FFPE sample is provided in a vessel such that dissociation occurs in said vessel. A non-solvent, aqueous solution can be provided in or added to the vessel with the FFPE sample, and paraffin can be subsequently disassociated from the paraffin-embedded sample by exposing the sample and non-solvent solution in the vessel to acoustic energy to disassociate paraffin from the sample. Biomolecules, such as nucleic acids, proteins and/or other components, can then be recovered from the aqueous portion of the sample after disassociation of paraffin. In one embodiment, dissociation can be performed one or more additional times on either the aqueous portion of a sample after a previous round of disassociation of paraffin or the aqueous portion of a sample as well as the tissue sample itself after a previous round of disassociation of paraffin. Recovery of the aqueous portion of any sample following an initial or subsequent round of disassociation can be by centrifuging and pipetting the processed suspension from the vessel or by pipetting liquid containing the biomolecules from the vessel. The recovered biomolecules may be subjected to any suitable further processing as desired, such as DNA purification processing using commercially available techniques and equipment or further focused acoustic treatment, for example, for additional processing (e.g., fragmenting of nucleic acids) and/or to enhance overall recovery of biomolecules. In some cases, the recovering step comprises centrifuging the tissue sample, thereby separating a supernatant solution containing nucleic acid material dissociated from insoluble contaminants. In some cases, the recovering step comprises purifying nucleic acid material by solid phase reversible immobilization (SPRI). Any SPRI compatible substrates (e.g., SPRI beads) known in the art can be used during a recovery step provided herein.

In one embodiment, the recovered biomolecules are not subjected to any further processing (e.g., fragmenting of nucleic acids) and instead are subjected to chromosomal conformation capture (e.g., Hi-C) methods as described herein.

In some cases, the disassociating step comprises exposing the FFPE sample to focused acoustic energy for a time sufficient to disassociate enough paraffin from the FFPE sample to allow recovery of the nucleic acid material and/or proteome material from the tissue sample. In some cases, the disassociating step comprises disassociating at least, more than or about 90%, 91%, 92%, 93%, 94%, 95%, 96%, 97%, 98%, 99%, 99.5% or 99.9% of paraffin attached to the FFPE sample. In some cases, the disassociating step comprises disassociating more than 90% of paraffin attached to the FFPE sample. In some cases, the disassociating step comprises disassociating more than 95% of paraffin attached to the FFPE sample. In some cases, the disassociating step comprises disassociating more than 98% of paraffin attached to the FFPE sample. In some cases, the disassociating step comprises disassociating more than 99% of paraffin attached to the FFPE sample. Performing one or more additional dissociation steps can increase the disassociation of paraffin attached to the FFPE sample by at least, at most or about 5%, 10%, 15%, 20%, 25%, 30%, 35%, 40%, 45% or 50% as compared to performing a single disassociation step. In some cases, the disassociating step comprises rehydrating the tissue sample while exposing the tissue sample to focused acoustic energy. In some cases, the disassociating step comprises maintaining a temperature of the solution at between 5° C. and 60° C. The solution may be at a temperature of about 18° C., to about 20° C., or a temperature of about 4° C. to about 7° C. The solution may be at a temperature of about 40° C., or a temperature of about 20° C., or a temperature of about 7° C. Thus, disassociation may be performed while the temperature of the sample is maintained below about 60° C., e.g., below about 45° C., below about 20° C., below about 10° C.

In some cases, the method further comprises maintaining the tissue sample in the vessel at below 50° C. until heating with sample to 90-100° C.

In some cases, the dissociating step comprises adding a protease (e.g., Proteinase K or trypsin) to the solution and the tissue sample in the vessel prior to exposing the tissue sample to focused acoustic energy. The processed sample and protease-containing solution may be exposed to focused acoustic energy a second time, e.g., for a period of 10-30 seconds (or more) to enhance the mixing of the protease with the sample and thereby enhance enzymatic activity. In one embodiment, acoustic treatment for 30 seconds or less (e.g., 10 seconds) may serve to suitably mix the protease with the sample prior to incubating the sample with the protease to further hydrolyze the proteins in the sample. Also, the inclusion of a glycerol material with the protease can be used to further enhance the enzyme activity and the effect of the acoustic energy as a driver of the protease action. This mixing treatment may be performed with the sample at a temperature of between 5-46° C., e.g., with the coupling medium 16 at about 46° C., about 20° C., about 7° C., although other temperatures are possible. In some cases, the method comprises inactivating the protease. In some cases, inactivating the protease comprises heating the vessel to about 98° C.

In one embodiment, the dissociating step comprises exposing the tissue sample (e.g., FFPE sample) to focused acoustic energy at an intensity suitable to avoid shearing the nucleic acid material. The majority of the fragments of nucleic acid material after exposing the tissue sample to focused acoustic energy in one or more disassociating steps can have a size of 1000 bp or greater. The nucleic acid material or the fragments of nucleic acid material can then be subjected to chromosomal conformation capture methods as provided herein.

The method and systems provided herein can further comprise repeating the dissociating step one or more times. In some cases, the method further comprises repeating the dissociating step while maintaining the vessel at about 4° C. to about 7° C. In some cases, the method further comprises repeating the dissociating step one or more times while maintaining the vessel at about 18° C. to about 20° C. followed by a final dissociating step while maintaining the vessel at about 4° C. to about 7° C. Similarly to the initial disassociation step, each additional disassociating step can be performed on tissue sample remaining in the vessel following a previous round of disassociation to which solution (e.g., non-solvent solution as described herein) is added. The final dissociating step is performed on the solution (e.g., aqueous solution) isolated from each previous round of disassociation.

In one embodiment, an acoustic treatment device is utilized in the dissociation steps present in the methods and system provided herein. The acoustic treatment device can include a vessel holding a formalin fixed, paraffin embedded tissue sample and a non-solvent, aqueous solution, and an acoustic energy source for providing acoustic energy to the sample while the sample is in the vessel and separated from the acoustic energy source. A vessel holder may support the vessel at a location at least partially in a focal zone of the acoustic energy, and a system control circuit may control the acoustic energy source to expose the sample to focused acoustic energy suitable to disassociate paraffin from the sample to allow recovery of biomolecules of the sample. The focused acoustic energy for use in the dissociation steps provided in the methods and systems provided herein can have a frequency of between about 100 kilohertz and about 100 megahertz. The focused acoustic energy can have a focal zone with a width of less than about 2 centimeters. The focused acoustic energy can originate from an acoustic energy source spaced from and exterior to the vessel (e.g., an acoustic treatment device), wherein at least a portion of the acoustic energy propagates exterior to the vessel. In some cases, the focused acoustic energy has a duty factor of between 10% and 30%. In some cases, the focused acoustic energy has a duty factor of about 15% or about 20%. In some cases, the focused acoustic energy has a peak intensity power of between 60W and 90W. In some cases, the focused acoustic energy has a peak intensity power of about 75W. In some cases, each disassociating step in any method provided herein is performed with a cycles per burst (cpb) of 200. In some cases, any of the methods provided herein that entails using focused acoustic energy to extract nucleic acid from a preserved sample (e.g., FFPE tissue sample) comprises at least one dissociating step such that the AFA is run for 5 min with a duty factor of 20%, a peak intensity of 75W and 200 cycles/burst in at least one of the dissociating steps. In some cases, the method provided herein comprises a first and a second dissociating step such that the first dissociating step is performed using AFA run for 5 min with a duty factor of 20%, a peak intensity of 75W and 200 cycles/burst, while the second dissociating step is performed using AFA run for 10 min with a duty factor of 15%, a peak intensity of 75W and 200 cycles/burst. In some cases, the method provided herein comprises more than two dissociating steps such that each dissociating step is performed using AFA run for 5 min with a duty factor of 20%, a peak intensity of 75W and 200 cycles/burst except for the final dissociating step, which is performed using AFA run for 10 min with a duty factor of 15%, a peak intensity of 75W and 200 cycles/burst.

In one embodiment, the dissociating step preserves formaldehyde crosslinks in the tissue sample. Further to this embodiment, the processed sample is then subjected to chromosomal conformational capture (e.g., Hi-C) and chromosomal structural variant identification (e.g., via sequencing) as described herein.

### Size Selection

Nucleic acid obtained from preserved (e.g., FFPE) biological samples can be fragmented to produce suitable fragments for analysis by chromosomal conformation capture methods provided herein. Template nucleic acids may be fragmented or sheared to desired length, using a variety of mechanical, chemical and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In some embodiments, nucleic acid from a biological sample is fragmented by sonication. In other embodiments, nucleic acid is fragmented by a hydroshear instrument. Generally, individual nucleic acid template molecules can be from about 2 kb bases to about 40 kb. In various embodiments, nucleic acids can be about 6 kb-10 kb fragments. In one embodiment, nucleic acid from a preserved tissue sample is fragmented using focused acoustic energy as described in WO2018195153, which is incorporated herein by reference.

In one embodiment, cross-linked DNA molecules may be subjected to a size selection step. Size selection of the nucleic acids may be performed to cross-linked DNA molecules below or above a certain size. Size selection may further be affected by the frequency of crosslinks and/or by the fragmentation method, for example by choosing a frequent or rare cutter restriction enzyme. In some embodiments, a composition may be prepared comprising crosslinking a DNA molecule in the range of about 1 kb to 5 Mb, about 5 kb to 5 Mb, about 5 kB to 2 Mb, about 10 kb to 2 Mb, about 10 kb to 1 Mb, about 20 kb to 1 Mb about 20 kb to 500 kb, about 50 kb to 500 kb, about 50 kb to 200 kb, about 60 kb to 200 kb, about 60 kb to 150 kb, about 80 kb to 150 kb, about 80 kb to 120 kb, or about 100 kb to 120 kb, or any range bounded by any of these values (e.g. about 150 kb to 1 Mb).

In some embodiments, sample polynucleotides are fragmented into a population of fragmented DNA molecules of one or more specific size range(s). In some embodiments, fragments can be generated from at least about 1, about 2, about 5, about 10, about 20, about 50, about 100, about 200, about 500, about 1000, about 2000, about 5000, about 10,000, about 20,000, about 50,000, about 100,000, about 200,000, about 500,000, about 1,000,000, about 2,000,000, about 5,000,000, about 10,000,000, or more genome-equivalents of starting DNA. Fragmentation may be accomplished by methods known in the art, including chemical, enzymatic, and mechanical fragmentation. In some embodiments, the fragments have an average length from about 10 to about 10,000, about 20,000, about 30,000, about 40,000, about 50,000, about 60,000, about 70,000, about 80,000, about 90,000, about 100,000, about 150,000, about 200,000, about 300,000, about 400,000, about 500,000, about 600,000, about 700,000, about 800,000, about 900,000, about 1,000,000, about 2,000,000, about 5,000,000, about 10,000,000, or more nucleotides. In some embodiments, the fragments have an average length from about 1 kb to about 10 Mb. In some embodiments, the fragments have an average length from about 1 kb to 5 Mb, about 5 kb to 5 Mb, about 5 kB to 2 Mb, about 10 kb to 2 Mb, about 10 kb to 1 Mb, about 20 kb to 1 Mb about 20 kb to 500 kb, about 50 kb to 500 kb, about 50 kb to 200 kb, about 60 kb to 200 kb, about 60 kb to 150 kb, about 80 kb to 150 kb, about 80 kb to 120 kb, or about 100 kb to 120 kb, or any range bounded by any of these values (e.g. about 60 to 120 kb). In some embodiments, the fragments have an average length less than about 10 Mb, less than about 5 Mb, less than about 1 Mb, less than about 500 kb, less than about 200 kb, less than about 100 kb, or less than about 50 kb. In other embodiments, the fragments have an average length more than about 5 kb, more than about 10 kb, more than about 50 kb, more than about 100 kb, more than about 200 kb, more than about 500 kb, more than about 1 Mb, more than about 5 Mb, or more than about 10 Mb.

In some embodiments, the fragmentation is accomplished mechanically comprising subjection sample DNA molecules to acoustic sonication. In some embodiments, the fragmentation comprises treating the sample DNA molecules with one or more enzymes under conditions suitable for the one or more enzymes to generate double-stranded nucleic acid breaks. Examples of enzymes useful in the generation of DNA fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mnt. In some embodiments, fragmentation comprises treating the sample DNA molecules with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample DNA molecules leaves overhangs having a predictable sequence. In some embodiments, the method includes the step of size selecting the fragments via standard methods such as column purification or isolation from an agarose gel.

### Chromosomal Structural Variants

The disclosure provides methods and systems for detecting one or more chromosomal structural variants in a subject.

As used herein, the term “chromosome” refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes. In humans, each chromosome has a short arm (termed “p” for “petit”) and a long arm (termed “q” for “queue”).

Each chromosome arm is divided into regions, or cytogenetic bands, that can be seen in a conventional karyotype using a microscope. The bands are labeled p1, p2, p3 etc. counting from the centromere out towards the telomeres. Higher-resolution sub-bands within the bands are sometimes also used to identify regions in the chromosome. Sub-bands are also numbered from the centromere out towards the telomere. Information on chromosome banding and chromosome nomenclature can be found in pp. 37-39 of Strachan, T. and Read, A. P. 1999. Human Molecular Genetics, 2nd ed. New York: John Wiley & Sons.

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity (e.g., an analogue of A will base pair with T. A polynucleotide of deoxyribonucleic acids (DNA) of specific identities and order is also referred to herein as a “DNA sequence.” Chromosomes comprise polynucleotides complexed with proteins (e.g. histones).

As used herein the terms “Structural Variant”, “Chromosomal Structural Variant”, “CSV” or “SV” refer to a difference in the structure of an individual's chromosome or chromosomes relative to the chromosome(s) in the genomes of other individuals within the same species or in a closely related species. Differences in chromosomal structure encompass differences in the arrangement and identity of DNA sequences in a chromosome. Differences in the arrangement of DNA sequences in a chromosome include both differences in the positions of DNA sequences on the chromosome relative to other sequences (e.g., translocations) and differences in orientation relative to other sequences (e.g., inversions). Differences in the identity of DNA sequences along a chromosome can include both new sequences and missing sequences, for example through the movement sequences from one chromosome to another non-homologous chromosome.

Chromosomal structural variations can be small or large in size, encompassing tens of base pairs, hundreds of base pairs, kilobases, megabases, or even significant portions (a half, a third or three-quarters, e.g.) of an individual chromosome. All size of chromosomal structural variations are within the scope of the disclosure.

There are multiple types of chromosomal structural variants, all of which are envisaged as within the scope of the methods and systems of the disclosure. Non-limiting examples of types of chromosomal structural variants include a translocation, a balanced translocation, an unbalanced translocation, a complex translocation, an inversion, a deletion, a duplication, a repeat expansion or a ring.

As used herein the term “translocation” refers to the exchange of DNA sequences between non-homologous chromatids, between two or more positions on the same chromatid, or between homologous chromatids that is not as a result of crossover during meiosis. Translocations can create gene fusions, which occur when two genes that are not normally adjacent to each other are brought into proximity. Alternatively, or in addition, translocations can disrupt gene function by breaking genes at the borders of the translocation. For example, a translocation can separate an open reading frame (ORF) from a distal regulatory element or bring the open reading frame into proximity with a new regulatory element, thereby affecting gene expression. Alternatively, or in addition, the break point of the translocation can occur in the middle of a gene, thereby creating a gene truncation. A “breakpoint” refers to the point or region of a chromosome at which the chromosome is cleaved during a translocation. A “breakpoint junction” refers to the region of the chromosome at which the different parts of chromosomes involved in a translocation join. Alternatively, or in addition, a translocation can affect the expression of one or more genes contained within the translocation by moving those genes to a new chromatin environment in the nucleus, for example by moving a DNA sequence from a region of strong gene expression (e.g. euchromatin) to a region of low gene expression (e.g. heterochromatin) or vice versa. Depending on the translocation, the translocation can have no effect on gene expression, can effect a single gene, or can effect multiple genes.

As used herein the term “balanced translocation” refers to the reciprocal exchange of DNA between non-homologous chromatids, or between homologous chromatids not as a result of crossover during meiosis. A “balanced translocation” is a translocation in which there is no loss of genetic material during the translocation, but all genetic material is preserved during the exchange. In an “unbalanced translocation” there is a loss of genetic material during the exchange.

As used herein, the term “reciprocal translocation” refers to a translocation which involves the mutual exchange of fragments between two broken chromosomes. In a reciprocal translocation, one part of one chromosome unites with the part of another chromosome.

As used herein, the terms “variant translocation”, “abnormal translocation” or “complex translocation” refer to the involvement of a third chromosome in a secondary rearrangement that follows a first translocation.

Translocations can be intrachromosomal (the rearrangement breakpoints occur within the same chromosome) or interchromosomal (the rearrangement breakpoints are between two different chromosomes).

As used herein, the term “inversion” refers to the rearrangement of DNA sequences within the same chromosome. Inversions change the orientation of a DNA sequence within a chromosome.

As used herein, the term “deletion” refers to a loss of a DNA sequence. Deletions can be any size, ranging from a few nucleotides to entire chromosomes. Translocations are frequently accompanied by deletions, for example at the translocation break points.

As used herein, the term “duplication” refers to a duplication of a DNA sequence (e.g., the genome contains three copies of a DNA sequence, instead of two). Duplications can be any size, ranging from a few nucleotides to entire chromosomes. Translocations are frequently accompanied by duplications.

As used herein, the term “repeat expansion” refers to tandem repeated sequences in the genome that with variable copy numbers between subjects. When there are a greater than average number of repeats of a repetitive sequence, the repetitive sequence has been expanded. Repeated sequences can comprise 2, 3, 4, 5, 6, 7, 8, 9, 10 or more repeated nucleotides. Expanded repeats are associated with a number of genetic disorders, including but not limited to Huntington's disease, spinocerebellar ataxias, fragile X syndrome, myotonic dystrophy, Friedreich's ataxia and juvenile myoclonic epilepsy.

All types of chromosomal structural variants can be identified using the methods and systems of the disclosure.

In some embodiments, the chromosomal structural variant identified by the methods and systems of the disclosure is a chromosomal variant that is known in the art. For example, the chromosomal structural variant identified by the methods of the disclosure is a chromosomal structural variant that has been previously described and characterized. Descriptions of chromosomal structural variants in the art include mapping one or more breakpoints of the chromosomal structural variant using techniques known in the art, for example by karyotyping, sequencing or Southern blot. In those embodiments wherein the chromosomal structural variant is known to cause a disease or disorder, descriptions of known chromosomal structural variants include clinical data such as symptoms, prognosis and recommended courses of treatment.

In some embodiments, the chromosomal structural variant identified by the methods and systems of the disclosure is a novel chromosomal variant. Novel chromosomal structural variants are variants that have not previously been described in the art. Novel chromosomal structural variants may be similar to chromosomal structural variants known in the art. For example, a chromosomal structural variant may be both recurrent, in that similar variants occur independently across multiple individuals, and novel, in that each individual with a recurrent variant comprises a variant with slightly different break points. In some embodiments, a novel chromosomal structural variant has one or more breakpoints that are similarly placed compared to a break point of a chromosomal structural variant known in the art. A similarly placed break point comprises a break point that is within 50 bp, within 100 bp, within 500 bp, within 1 kb, within 5 kb, within 10 kb, within 20 kb, within 50 kb, within 100 kb, within 200 kb or within 500 kb or within 1 Mb of a break point of a chromosomal structural variant known in the art. In some embodiments, a novel chromosomal structural variant has one or more breakpoints that are identical to a break point of a chromosomal structural variant known in the art, and one or more breakpoints that are not identical to a break point of a chromosomal structural variant known in the art. In some embodiments, a novel chromosomal structural variant does not have similar or identical break points to a chromosomal structural variant known in the art.

### Representation of Chromosomal Structural Variants

The disclosure provides systems and methods for detecting one or more chromosomal structural variants in a subject, and representing the chromosomal structural variant or variants in a manner that can be readily interpreted by a person of ordinary skill in the art (for example, a clinician, a doctor, a patient or a researcher).

In some embodiments, the chromosomal structural variant is represented as a karyotype. Karyotyping is a traditional method used to identify chromosomal structural variants. In karyotyping, the development of cells is arrested during metaphase, bound chromatids are extracted, stained and photographed, and the structural properties of the chromatids are mapped using the cytogenetic banding patterns of the chromosome. Karyotyping is expensive, time consuming and of limited resolution. Traditional karyotyping relies on the cytogenetic bands and sub bands within the karyotype to map the boundaries of chromosomal structural variants, and so cannot resolve chromosomal structural variants that are finer (smaller) than the cytogenetic bands of the karyotype, which typically have a minimum resolution of about 5 Mb. In contrast, the systems and methods of the disclosure are able to achieve a resolution that is at least 1,000 finer than a traditional karyotype.

Traditional karyotype results can be represented as karyotype spreads, which are images of all the chromosomes analyzed in the karyotype, stained to identify cytogenetic bands and arranged in ordered pairs. While the methods of the disclosure provide a resolution superior to a traditional karyotype, the chromosomal structural variants identified by the methods of the disclosure can be represented as a karyotype or karyotype spread. This facilitates interpretation of chromosomal structural variant data of the disclosure by doctors and clinicians, who may be more familiar with and trained to identify chromosomal structural variants based on traditional karyotypes.

In some embodiments, chromosomal structural variants of the disclosure are represented as a karyotype.

### Clinical Chromosomal Structural Variants

The disclosure provides methods and systems for detecting one or more chromosomal structural variants in a subject, and further relating the one or more chromosomal structural variants to relevant biological information. Relevant biological information includes, but is not limited to, the clinical significance of the variant, associated diseases or disorders, symptoms thereof, associated genes and/or genetic mutations, effects of the chromosomal structural variant on gene expression, and recommended courses of treatment or therapies.

In some embodiments, the chromosomal structural variants that are identified by the systems and methods of the disclosure cause one or more diseases or disorders.

In some embodiments, the chromosomal structural variants that cause diseases or disorders are inherited, i.e. the chromosomal structural variant is transmitted from parent to offspring via the germ line. All inherited chromosomal structural variants are within the scope of the systems and methods of the disclosure.

In other alternative embodiments, the chromosomal structural variants that cause diseases or disorders are somatic, i.e. the chromosomal structural variant arise de novo in a cell in the individual. Depending upon when in development a somatic chromosomal structural variant arises, somatic chromosomal structural variants can occur all the cells in an organism (the chromosomal structural variant arises prior to the first cell division), or can occur in a subset of the cells in the organism (the chromosomal structural variant occurs later in development, or in an adult). Exemplary disorders that can occur in every cell include aneuploidies such as Turner syndrome (X chromosome monosomy) and Down syndrome (trisomy 21).

Exemplary disorders caused by haploinsufficiencies resulting from deletions include Williams syndrome, Langer-Giedion syndrome, Miller-Dieker syndrome, and DiGeorge/velocardiofacial syndrome. All somatic chromosomal structural variants are within the scope of the systems and methods of the disclosure.

In some embodiments, the diseases or disorders caused by chromosomal structural variants are caused by a chromosomal structural variant that occurs de novo in the subject. In some embodiments, the chromosomal structural variant that occurs de novo is a recurrent structural variant. Many chromosomal structural variants are recurrent, in that the same or similar chromosomal structural variants occur de novo in multiple individuals. These individuals are not necessarily related. In many cases, the recurrent chromosomal structural variants are caused by non-allelic homologous recombination mediated by flanking segmental duplications. In non-allelic homologous recombination, improper crossing over between non-homologous DNA sequences, for example DNA sequences that contain similar repetitive DNA sequences, leads to a tandem or direct duplication and a deletion. Non-limiting examples of diseases and disorders caused by recurrent chromosomal structural variants include in Charcot Marie Tooth disease, hereditary neuropathy with liability to pressure palsies, Prader Willi, Angelman, Smith Magenis, DiGeorge/velocardiofacial (DGS/VCFS), Williams Beurens, and Sotos syndromes.

Databases of chromosomal structural variants are well known to persons of ordinary skill in the art. For example, biological information regarding chromosomal structural variants and their associated diseases and disorders, and treatments for these diseases and disorders can be found in the Online Mendelian Inheritance in Man (omim.org), the Mitelman Database of Chromosome Aberration and Gene Fusion in Cancer (cgap.nci.nih.gov/Chromosomes/Mitelman) and the NCBI database (ncbi.nlm.nih.gov/clinvar?term=300005[MIM]).

Chromosomal structural variants and associated diseases and disorders are also described by the National Institute of Health's Genetic and Rare Diseases Information Center (rarediseases.info.nih.gov/diseases/diseases-by-category/36/chromosome-disorders).

In some embodiments, chromosomal structural variants do not occur in every cell in a tissue of the subject. In some embodiments, the cells with the chromosomal structural variant(s) are cancer cells in the subject. A subject with a cancer can have cancer cells with one or more chromosomal structural variants, while the non-cancerous cells of the subject do not have a chromosomal structural variant, or do not have the same chromosomal structural variants that are seen in the cancer cells of the subject.

Cancers are diseases caused by the proliferation of malignant neoplastic cells, such as tumors, neoplasms, carcinomas, sarcomas, blastomas, leukemias, lymphomas and the like. For example, cancers include, but are not limited to, mesothelioma, leukemias and lymphomas such as cutaneous T-cell lymphomas (CTCL), non-cutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.) and liver cancer.

Most cancers acquire one or more clonal chromosomal structural variants during the development of the cancer, which can be identified by the systems and methods of the disclosure. In many cases, recurrent chromosomal structural variants are associated with particular morphological and clinical disease characteristics. Structural variants in cancer cells can affect the expression and/or function of proto-oncogenes and tumor suppressors. Structural variants in cancer cells can also facilitate the progression of the cancer itself, as mutations and changes in gene expression caused by the chromosomal structural variant(s) promote increased growth and invasiveness of tumor cells, and tumor vascularization. Identifying the specific chromosomal structural variants in a cancer cells in a cancer sample allows for the more effective selection of cancer therapies. These therapies can be tailored to changes in gene expression and cancer pathologies associated with the particular chromosomal structural variants in the cancer cells. Thus, the rapid and effective identification of chromosomal structural variants in cancers is a critical piece of the cancer diagnostic and treatment arsenal.

In some embodiments, structural variants in cancer cells create novel fusion proteins which promote the progression of the cancer. A non-limiting, exemplary list of chromosomal structural variants that cause fusion proteins associated with cancers is described in Hasty, P. and Montagna, C. (2014) Mol. Cell. Oncol.: e29904. Currently there are 21,477 documented gene fusions and 69,134 cases documented in the Cancer Genome Anatomy Project (cgap.nci.nih.gov/Chromosomes/Mitelman), all of which are envisaged as falling within the scope of the instant disclosure.

In some embodiments, chromosomal structural variants in cancer cells lead to changes in gene regulation and gene expression, which contribute to the progression of the cancer. A chromosomal structural variant can lead to the downregulation of one or more the tumor suppressors, which are genes that protect the cell from cancer. For example, a chromosomal structural variant with a break point near a tumor suppressor can separate the coding sequence of the tumor suppressor from a regulatory element. Alternatively, or in addition, a chromosomal structural variant can lead to the conversion of one or more proto-oncogenes into an oncogene which promotes cancer progression. For example, a chromosomal structural variant with a break point near a proto-oncogene can bring the proto-oncogene into proximity of a novel regulatory element, leading to upregulated expression. Exemplary tumor suppressors that can be down regulated by the chromosomal structural variants of the disclosure include, but are not limited to, p53, Rb, PTEN, INK4, APC, MADR2, BRCA1, BRCA2, WT1, DPC4 and p21. Exemplary oncogenes that can be upregulated by the chromosomal structural variants of the disclosure include, but are not limited to, Abl1, HER-2, c-KIT, EGFR, VEGF, B-Raf, Cyclin D1, K-ras, beta-catenin, Cyclin E, Ras, Myc and MITF. All chromosomal structural elements which affect proto-oncogenes and tumor suppressor genes are envisaged as within the scope of the systems and methods of the disclosure.

### Chromosomal Conformation Capture

Provided herein are systems and methods that use chromosomal conformation capture techniques to identify one or more chromosomal structural variants in a subject.

The terms “chromosomal conformational capture” and “chromosome conformation analysis” are used interchangeably herein.

The methods of the disclosure can use standard chromatin conformation data, such as Hi-C data, generated from a tissue sample (e.g. cancerous or normal tissues or cells) or preserved tissue sample (e.g., FFPE sample). The computational methods involves the training of one or more classifiers, which can be used in more than one of the major applications. The set of classifiers chosen may include deep learning models, gradient descent models, graph network models, neural network models, support vector machine models, expert system models, decision tree models, logistic regression models, clustering models, Markov models, Monte Carlo models, or other machine learning models, as well as models which fit observed data to probabilistic models such as likelihood models. The set of classifiers can be trained by labeled or unlabeled data, which can be generated from real biological samples, simulated genomes which may have simulated mutations, or generated by another algorithm, such as algorithms used in a generative adversarial network. The training data consists of chromatin conformation data or data derived from it (such as a contact matrix, and may be normalized, filtered, compressed, or smoothed) and clinical or biological information about the effects, properties, implications, or outcomes associated with the data.

In some embodiments of the systems and methods of the disclosure utilize one or more classifiers that are trained using chromosomal conformation capture data. In some embodiments, the one or more classifiers are trained using experimentally determined chromosomal conformational capture data. In some embodiments, the one or more classifiers are trained using simulated chromosomal conformational capture data. In some embodiments, the one or more classifiers are trained using a combination of experimentally determined and simulated chromosomal conformational capture data.

In some embodiments, the chromosomal conformational capture data used to train the one or more machine learning classifiers comprises experimentally determined chromosomal conformational capture data. In some embodiments, the experimentally determined chromosomal conformational capture data comprises a plurality of sets of reads from healthy subjects. In some embodiments, the experimentally determined chromosomal conformational capture data comprises a plurality of sets of reads from subjects with known chromosomal structural variants.

Chromosomal conformational data is generated by chemically cross-linking regions of the genome that are in close spatial proximity. In one embodiment, the crosslinking for chromosomal conformational capture or proximity ligation is essentially the same as is generated during the formalin fixation of solid tissues for histology, thereby making Hi-C compatible with FFPE tissues. Subsequently, the cross-linked chromatin can be fragmented. The fragments can be ligated together to create chimeric sequences which can be detected using any sequence detection method known in the art, such as, for example, CHIP analysis, PCR analysis or sequencing (e.g., Illumina paired end chemistry). Sequencing these chimeric DNA molecules can capture the signal of long-range chromatin interactions (such as promoter-enhancer interactions). The signal in proximity ligation sequencing can also reflect the linear distance between two sequences on a chromosome.

In one embodiment, the methods and systems provided herein that utilize FFPE tissue samples, utilize the cross-linking performed during preparation of the FFPE sample for chromosomal conformational capture. The cross linked nucleic acid (e.g., DNA) can then be fragmented and ligated to generate chromatin/nucleic acid (e.g., DNA) complexes for subsequent sequence detection. In one embodiment, the cross linked nucleic acid (e.g., DNA) is restriction enzyme digested and ligated to generate chromatin/nucleic acid (e.g., DNA) complexes which are identified by high-throughput sequencing. In one embodiment, the restriction enzyme used to digest the cross-linked nucleic acid (e.g., DNA) during chromosomal conformational capture is DpnII. The resultant sequence detected (e.g., sequence reads) can be mapped to a genome, for example a reference genome, to determine the frequency with which each interaction occurs within the population of cells that was used to generate the initial sample. When two loci are in close spatial proximity, they can generate more reads that comprise DNA sequences that map both loci than if the two loci are not in close spatial proximity.

Experimentally determined chromosomal conformational capture data may form part of an input file used by a system to carry out the methods described herein. The set of reads may be generated by any suitable method based on chromatin interaction techniques or chromosome conformation analysis techniques. Chromosome conformation analysis techniques that may be used in accordance with the embodiments described herein may include, but are not limited to, Chromatin Conformation Capture (3C), Circularized Chromatin Conformation Capture (4C), Carbon Copy Chromosome Conformation Capture (5C), Chromatin Immunoprecipitation (ChIP; e.g., cross-linked ChIP (XChIP), native ChIP (NChIP)), ChIP-Loop, genome conformation capture (GCC) (e.g., Hi-C, 6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (e.g. Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C and Hybrid Capture Hi-C. In some embodiments, the dataset is generated using a genome-wide chromatin interaction method, such as Hi-C.

In some embodiments, chromosomal conformational data can be generated from a population of cells. In some embodiments, chromosomal conformational capture data is generated by Chromatin Conformation Capture (3C). 3C is used to analyze the organization of chromatin in a cell by quantifying the interactions between genomic loci that are nearby in 3-D space. 3C quantifies interactions between a single pair of genomic loci. In some embodiments, chromosomal conformational capture data is generated by Circularized Chromatin Conformation Capture (4C). 4C captures interactions between one locus and all other genomic loci. In some embodiments, chromosomal conformational capture data is generated by Carbon Copy Chromosome Conformation Capture (5C). 5C detects interactions between all restriction fragments within a given region. In some embodiments, the region is one megabase or less. In some embodiments, chromosomal conformational capture data is generated by Chromatin Immunoprecipitation (ChIP; e.g., cross-linked ChIP (XChIP), native ChIP (NChIP)). In some embodiments, chromosomal conformational capture data is generated by ChIP-Loop. In some embodiments, chromatin immunoprecipitation based methods incorporate chromatin immunoprecipitation (chIP) based enrichment and chromatin proximity ligation to determine long range chromatin interactions. In some embodiments, chromosomal conformational capture data is generated by Hi-C. Hi-C uses high-throughput sequencing to find the nucleotide sequence of fragments that map to both partners in all interacting pairs of loci. In some embodiments, chromosomal conformational capture data is generated by Capture-C. Capture-C selects and enriches for genome-wide, long-range contacts involving active and inactive promoters. In some embodiments, chromosomal conformational capture data is generated by SPLiT-seq. SPLiT-seq is a technique that can be used to transcriptome profile single cells. In some embodiments, chromosomal conformational capture data is generated by Nuclear Ligation Assay (NLA). Similar to 3C, NLA can be used to determine the circularization frequencies of DNA following proximity based ligation. In some embodiments, chromosomal conformational capture data is generated by Concatamer Ligation Assay (COLA). COLA is a Hi-C based protocol that uses the CviJI restriction enzyme to digest chromatin. In some embodiments, using COLA results in smaller fragments compared to traditional Hi-C. In some embodiments, chromosomal conformational capture data is generated by Cleavage Under Targets and Release Using Nuclease (CUT& RUN). CUT & RUN uses a targeted nuclease strategy for high-resolution mapping of DNA binding sites. For example, CUT&RUN can use an antibody-targeted chromatin profiling method in which a nuclease tethered to protein A binds to an antibody of choice and cuts immediately adjacent DNA, releasing DNA bound to the antibody target. CUT & RUN can be carried out in situ. CUT & RUN can produce precise transcription factor or histone modification profiles, as wells as mapping long-range genomic interactions. In some embodiments, chromosomal conformational capture data is generated by DNase Hi-C. DNase Hi-C uses DNase I for chromatin fragmentation, and can overcome restriction enzyme related limitations in conventional Hi-C protocols. In some embodiments, chromosomal conformational capture data is generated by Micro-C. Micro-C using micrococcal nuclease to fragment chromatin into mononucleosomes. In some embodiments, chromosomal conformational capture data is generated by Hybrid Capture Hi-C. Hybrid Capture Hi-C combines targeted genomic capture and with Hi-C to target selected genomic regions.

In some alternative embodiments, chromosomal conformational capture data can be generated from a single cell. For example, the chromosomal conformation capture data can be generated using Single-cell Hi-C (scHi-C) or Combinatorial Single-cell Hi-C. Single-cell Hi-C is an adaptation of Hi-C to single-cell analysis by including in-nucleus ligation. Combinatorial single-cell Hi-C is a modified single-cell Hi-C protocol that adds unique cellular indexing to measure chromatin accessibility in thousands of single cells per assay.

In some embodiments, chromosomal conformational capture data can be generated from a proximity ligation based protocol that is carried out in situ, i.e. in intact nuclei.

In some embodiments, chromosomal conformational capture data can be generated from a proximity ligation based protocol that is carried out in vitro. Exemplary in vitro based protocols include Chicago® from Dovetail Genomics, which using high molecular weight DNA as a starting material. In some embodiments, the input DNA is about 20-200 kbp. In some embodiments, the input DNA is about 50 kbp.

In one embodiment, generation of chromosome conformation capture data from nucleic acid material isolated from a preserved tissue sample obtained from a subject comprises: proximity ligating the nucleic acid material to form a library of proximity-ligated polynucleotides and identifying paired polynucleotide sequences in the library of proximity-ligated polynucleotides.

In one embodiment, generation of chromosome conformation capture data from nucleic acid material isolated from a preserved tissue sample obtained from a subject comprises: fragmenting the nucleic acid material, proximity ligating the nucleic acid material to form a library of proximity-ligated polynucleotides, and identifying paired polynucleotide sequences in the library of proximity-ligated polynucleotides.

The identifying step can comprise any method known in the art for identifying or detecting specific sequences such as, for example, PCR, CHIP or sequencing analysis. In one embodiment, the identifying step entails sequencing the proximity ligations in order to generate chromosomal conformational capture data.

Chromosomal conformational capture data can be generated using any sequencing methods or next generation sequencing platform known in the art. For example, chromosomal conformational capture data may be generated by proximity ligation followed by sequencing on an Oxford Nanopore machine (Pore-C), a Pacific Biosciences machine (SMRT-C), a Roche/454 sequencing platform, ABI/SOLiD platform, or an Illumina/Solexa sequencing platform.

In some embodiments of the systems and methods of the disclosure further comprise mapping reads generated by chromosomal conformational capture onto a genome. In some embodiments, the sets of reads may be aligned with the genome any suitable alignment method, algorithm or software package known in the art. Suitable short read sequence alignment software that may be used to align the set of reads with an assembly include, but are not limited to, BarraCUDA, BBMap, BFAST, BLASTN, BLAT, Bowtie, HIVE-hexagon, BWA, BWA-PSSM, BWA-mem, CASHX, Cloudburst, CUDA-EC, CUSHAW, CUSHAW2, CUSHAW2-GPU, CUSHAW3, drFAST, ELAND, ERNE, GASSST, GEM, Genalice MAP, Geneious Assembler, GensearchNGS, GMAP and GSNAP, GNUMAP, IDBA-UD, iSAAC, LAST, MAQ, mrFAST and mrsFAST, MOM, MOSAIK, Novoalign & NovoalignCS, NextGENe, NextGenMap, Omixon, PALMapper, Partek, PASS, PerM, PRIMEX, QPalma, RazerS, REAL, cREAL, RMAP, rNA, RTG Investigator, Segemehl, SeqMap, Shrec, SHRIMP, SLIDER, SOAP, SOAP2, SOAP3, SOAP3-dp, SOCS, SSAHA, SSAHA2, Stampy, SToRM, subread and Subjunc, Taipan, UGENE, VelociMapper, XpressAlign, and Zoom.

In some embodiments of the systems and methods of the disclosure further comprise filtering out reads that align poorly to a reference genome prior to applying classifiers for detecting or predicting a likelihood that the subject from which the sample (e.g., preserved tissue sample) was obtained has a known chromosomal structural variant(s). The classifier can be any classifier known in the art for predicting such a likelihood. In one embodiment, the classifier is any classifier described in U.S. 62/825,499 filed on Mar. 28, 2019. In some embodiments, the method comprises filtering out reads that align poorly in a training dataset. In some embodiments, the method comprises filtering out reads that align poorly in the data from the subject. In some embodiments, filtering out reads comprises mapping the chromosomal conformational capture reads onto a reference genome and filtering out the low quality alignment data. For example, reads can be aligned to a reference genome using BWA-mem, and low quality alignment data with less than MQ 20 is excluded.

### Machine Learning Classifiers

Disclosed herein are methods of treating a subject with a chromosomal structural variant comprising: (a) receiving a test set of reads from a sample from the subject; (b) aligning the test set of reads from the subject to a reference genome; (c) training a classifier to distinguish between sets of reads from healthy subjects and sets of reads corresponding to known chromosomal structural variants; (d) applying the classifier to the mapped set of reads from the subject; (e) computing a likelihood that the subject has a known chromosomal structural variant; and (f) generating a karyotype of the subject; wherein the test set of reads, the sets of reads from healthy subjects and the sets of reads corresponding to known chromosomal structural variants are generated by a chromosome conformation analysis technique.

In some embodiments, the classifier is selected from the group consisting of a deep learning model classifier, a gradient descent model classifier, a graph network model classifier, a neural network model classifier, a support vector machine, an export system model classifier, a decision tree model classifier, a logistic regression model classifier, a clustering model classifier, a Markov model, a Monte Carlo model or a likelihood model classifier.

In some embodiments, the classifier is a likelihood model classifier. Likelihood model classifiers are a type of supervised machine learning classifier.

The disclosure provides methods of training a likelihood model classifier comprising (i) importing a plurality of sets of reads from healthy subjects into the classifier; (i) importing a plurality of sets of reads corresponding to known chromosomal structural variants into the classifier; (iii) representing each known chromosomal structural variant as a bounding rectangle comprising a start and an end location in a genome of the chromosomal structural variant, and a label; (iv) partitioning the sets of reads from (i) and (ii) by genomic location; (v) transforming the partitioned sets of reads from (iv) into a geometric data structure; (vi) modeling a frequency of links between any two genomic locations for each of the sets of reads from (i) and (ii) using a negative binomial distribution model; and (vii) training the negative binomial distribution model to recognize a null distribution from the plurality of sets of reads from healthy subjects, wherein the negative binomial distribution model is trained to recognize a null distribution at the bounding rectangle of each known chromosomal structural variant.

The classifier is trained by importing labeled training data. In some embodiments, the training data comprises a representation of each known chromosomal structural variant as a bounding rectangle comprising a start and an end location in a genome of the chromosomal structural variant, and a label. In some embodiments, the training data comprises a plurality of sets of reads from healthy subjects and a plurality of sets of reads corresponding to known chromosomal structural variants. The sets of reads can be simulated, experimentally determined, or a mixture of both. In some embodiments, the sets of reads from healthy subjects comprise reads corresponding to the genomic locations of each known chromosomal structural variant. This allows the classifier to model the distribution of linkage frequencies for the null distribution (no CSV) for all the locations of all known chromosomal structural variants. In some preferred embodiments, the training data comprises sets of reads that are independent and identically distributed. In some embodiments, the imported training data is partitioned by genomic location, and transformed into geometric data structure such as a 2-d k-d tree or a matrix.

In some embodiments, a certain probability distribution in the testing data from the subject is assumed and its required parameters (e.g. probability model) are calculated during the training phase. In some embodiments, the probability model used by the classifier is determined by the training data. Exemplary probability models include Bernoulli models, binomial models, negative binomial models, multinomial models, Gaussian models or Poisson distributions.

In some embodiments, the probability model comprises a negative binomial distribution. Negative binomial distributions are advantageous over other models in that it can account for over-dispersion of read count data.

In the learning phase of the classifier, the input is the training data and the output is the parameters that are required for the classifier. Exemplary parameters include maximum likelihood Estimation (MLE), Bayesian estimation (maximum a posteriori) or optimization of loss criterion.

Following training, the likelihood model classifier is applied to a mapped set of chromosomal conformational capture reads from a subject. In some embodiments, applying the likelihood model classifier comprises fitting the transformed and partitioned test set of reads from the subject to the null model and to an alternate model for each known chromosomal structural variant. In some embodiments, the null model is the distribution of linkage frequencies seen in a subject that does not have a known chromosomal structural variant. In fitting to the null model, the likelihood model classifier identifies known chromosomal structural variants by looking for the absence of the null model, which is the distribution of linkages frequencies between every pair of loci found in a healthy subject, rather than looking for the presence of a known chromosomal structural variant. In some embodiments, fitting the transformed and partitioned test set of reads from the subject to the null model comprises fitting across the entire genome. In some alternative embodiments, the fitting comprises fitting across a portion of the genome corresponding to the bounding rectangle of each known chromosomal or subchromosomal structural variant.

In some embodiments, the methods comprise computing a likelihood ratio of the fit of the transformed and partitioned test set of reads to the null model versus the alternative models for each known chromosomal structural variant. Likelihood ratio tests are statistical tests used for comparing the goodness of fit of two statistical models, a null model (no CSV) and an alternative model (the presence of a known CSV). The test is based on the ratio of likelihoods of the two models, and expresses how many times more likely the data are under one model over the other model. Methods of computing likelihood or log-likelihood ratios, or transformations of these ratios scaled by constant factors, are well known to persons of ordinary skill in the art. In some embodiments, a proximity signal is represented in a matrix, or in rectangular subregions of the matrix can be further subdivided into quadrants about a focal coordinate (x, y). In some embodiments, the data in the matrix is binned. In such embodiments, a theoretical model can be developed to describe the changes in proximity signal expected for various structural variants, including balanced translocations, unbalanced translocations, inversions, insertions, deletions, or other copy number variations. Such theoretical models can include the use of beta, gamma, binomial, negative binomial, bimodal, multimodal, empirically fitted spline, Poisson, Dirichlet, uniform, linear, quadratic, polynomial, exponential, logarithmic, triangle, power law, Bayesian, or other suitable distributions, or any combination thereof, to model proximity signal or the apportionment thereof among regions which would theoretically be on the same chromosome, be on different chromosomes, be on the same chromosome with a given distance or range of distances between them, be on the same chromosome with a given relative arrangement, or have any other theoretical structural arrangement relative to each other. In such embodiments, theoretical models may be trained based on data in a single sample, trained against a multi-sample training set, or tuned using human-configured or fixed parameters. In such embodiments, the likelihood of a given theoretical model being present and centered on the focal coordinate can be calculated by measuring the likelihood of the observed data given the model. In such embodiments, a series of such theoretical models, reflecting the expected proximity signal of various types of structural variations being present, can be tested against observed proximity signal in a given region, and a region can be scanned for possible variant calls at various focal coordinates using maximum likelihood gradient descent, the Nelder-Mead method, the Broyden-Fletcher-Goldfarb-Shanno (BFGS) method, binary search, exhaustive search, entropy minimization techniques, or any other suitable optimization or minimization technique. In such embodiments, multiple theoretical models can be compared to combinations of focal points to identify more than one structural variant in a given region, yielding sets of fitted models that represent specific called variants at specific focal coordinates. In such an embodiment, fitted models may be weighted using Akaike information criterion (AIC), Bayesian information criterion (BIC), deviance information criterion (DIC), or any other suitable information criterion measure, in order to select the most likely combination of focal coordinates and called variants to have produced the observed data, thereby controlling for natural variation, background, or noise in the proximity signal and reducing the possibility of false positive or false negative variant calls. In some embodiments, the subject is determined to have a known chromosomal structural variant when the likelihood ratio for that known chromosomal variant is less than 0.5, 0.45, 0.40, 0.35, 0.30, 0.25, 0.20, 0.15, 0.10, 0.09, 0.08, 0.07, 0.06, 0.05, 0.04, 0.03, 0.02, 0.01, 0.009, 0.008, 0.007, 0.006, 0.005, 0.003, 0.002, 0.001, 0.0009, 0.0008, 0.007, 0.006, 0.005, 0.0004, 0.0003, 0.0002 or 0.0001. In some embodiments, the likelihood ratio is greater than 75%, 80%, 85%, 90%, 95%, 96%, 97, 98%, 99%, 99.1%, 99.2%, 99.3%, 99.4%, 99.5%, 99.6%, 99.7%, 99.8% or 99.9%. In some embodiments, the likelihood ratio is expressed as a log likelihood ratio.

The disclosure provides methods of detecting chromosomal structural variants in a subject comprising: (a) training a first classifier to detect at least one region of a first contact matrix comprising at least one chromosomal structural variant; (b) importing a first contact matrix from a subject into the first classifier, wherein the contact matrix is produced by a chromosome conformation analysis technique; (c) applying the first classifier to the first contact matrix to detect at least one region of the first contact matrix containing at least one chromosomal structural variant; (d) expressing each chromosomal structural variant identified by the first classifier as a bounding box comprising a start and an end in a genome, and a label; (e) training a second classifier to relate the at least one chromosomal structural variant to biological information; (f) importing the bounding box and the label of the at least one chromosomal structural variant identified by the first classifier into the second classifier; and (g) applying the second classifier; thereby identifying each chromosomal structural variant of the subject and the biological information related to each chromosomal structural variant. In some embodiments, the method further comprises after step (d) and before step (e): (i) generating an second contact matrix, wherein the second contact matrix comprises the start and end genomic locations of the bounding box, and wherein a resolution of the second contact matrix is finer than a resolution of the first contact matrix; (ii) applying the first classifier to the second contact matrix to detect at least one region of the second contact matrix containing the at least one chromosomal structural variant; and (iii) expressing the at least one chromosomal structural variant as a second bounding box comprising a start and an end genomic location of the at least one chromosomal structural variant, and the label, wherein the second bounding box comprises a higher resolution than the bounding box.

In some embodiments, the first classifier comprises a convolutional neural network (CNN). CNNs are a class of deep neural networks frequently used to analyze visual imagery. CNNs of the disclosure take an input contact matrix and assign importance (learnable weights and biases) to various aspects/objects in the contact matrix and be able to differentiate between contact matrices from datasets with and without chromosomal structural variants and the type and positions of the variants. The architecture of CNNs is designed to mimic that of neural networks in the human brain. In some embodiments, the CNN captures relationships in a contact matrix by the application of a series of filters.

In some embodiments, the CNN is trained on contact matrices generated from simulated and biological samples. In some embodiments, training the CNN comprises: (i) importing a first training dataset into the CNN, wherein the training dataset comprises contact matrices generated from simulated and biological samples; (ii) using transfer learning to apply a pre-trained model to the CNN; and (iii) re-training the CNN with a second training dataset, wherein the second training dataset consists of contact matrices from biological samples. In some embodiments, the first training dataset comprises or consists of contact matrices from subjects that do not have chromosomal structural variants. In alternative embodiments, the first training dataset comprises at least one contract matrix form a subject with a chromosomal structural variant. In further alternative embodiments, the first training dataset comprises contact matrixes comprising a plurality of chromosomal structural variants. In some embodiments, the first training dataset comprises full genome contract matrices and contact matrices consisting of portions of genomes.

“Transfer learning”, as used herein, refers to a process in machine learning wherein a model developed for a first task is re-used as a starting point for developing a model for a second task. Applying transfer learning saves time and computing power when training neural networks. Methods for applying transfer learning to CNNs will be readily apparent to one of ordinary skill in the art.

In some embodiments, the second classifier comprises a recurrent neural network, a sense detector or a k-nearest neighbors model, all of which will be known to a person of ordinary skill in the art.

In some embodiments, the second classifier comprises as sense detector. A sense detector, also sometimes referred to as a text classifier, is a type of machine learning classifier that is trained, and used, to classify text based on meaning. There are many machine learning classifiers that can be trained as sense detectors, including, but not limited to Naive Bayes, Support Vector machines, Deep learning, convolutional neural networks, recurrent neural networks and hybrid systems that combine machine learning and rule based systems.

Recurrent neural networks are a class of artificial neural networks where connections between nodes in the network form a directed graph along a temporal sequence. Loops between the nodes allow information to persist in the network.

A k-nearest neighbors model is a type of machine learning model that is used to classify and regress data. A k-nearest neighbors model is able to identify what category or categories data belongs in, and also estimate the relationships amongst variables in a dataset. In some embodiments, the k-nearest neighbors model is supervised machine learning model that is trained on a training dataset.

In some embodiments, the sense detector is trained using clinical label data from known chromosomal structural variations, diagnosis data, clinical outcome data, drug or treatment response data or metabolic data. Sources of such data are readily known to persons of ordinary skill in the art.

### Methods of Treatment

Provided herein are methods of treating a subject with a disease or disorder caused by a chromosomal structural variant. The methods comprise identifying a chromosomal structural variant using the systems and methods of the disclosure, associating the identified chromosomal structural variant with relevant biological information, recommending a course of treatment, and administering the treatment to the subject.

By comprehensively identifying chromosomal structural variants and relating these variants to diseases and disorders and treatment methods, the systems and methods of the disclosure allow clinicians and doctors to tailor treatments to individual subjects. For example, chromosomal structural variants found in some cancers are associated with better or worse clinical outcomes for particular cancer therapies. In one specific example, methods of the disclosure can be used to identify breast cancers with copy number increases in ERBB2 (epidermal growth factor receptor 2, or HER2), which can be targeted with EGFR inhibitors as part of a recommended course of treatment. Further examples of targeted cancer therapies are shown in Table 1 below:

TABLE 1
Genes and pathways affected by chromosomal
structural variants and targeted therapies.

| Target | Pathway | Agents |
|---|---|---|
| ERBB2 (HER2) | RAS/Raf/MAPK | trastuzumab, pertuzumab, |
| | and PI3K/Akt | apatinib, afatinib, neratinib |
| EGFR | PI3K/Akt | erlotinib, gefitinib. |
| | | dacomitinib, neratinib, |
| | | simertinib, rociletinib, |
| | | olmutinib |
| FLT3-ITD | STAT, ERK, AKT, | sorafenib, daunoribuicin, |
| | C-Myc | cytarabine |
| VEGF and | VEGF and mTOR | sorafenib, sunitinib, |
| mTOR | | pazopanib, bevacizumab, |
| | | temsirolimus, everolimus |
| VEGFR | Ras/Raf/MEK/ERK | sorafenib, dovitinib |
| BCR-Abl | | imatinib, nilotinib, dasatinib, |
| | | bosutinib, ponatinib, |
| | | bafetinib |

Any chromosomal structural variant that causes a disease or disorder falls is envisaged as within scope of the disorder.

Any chromosomal structural variant that causes a disease or disorder with a recommended treatment regimen falls is envisaged as within scope of the disorder.

### EXAMPLES

### Example 1-Method for Extracting Nucleic Acid from FFPE Using Adaptive Focused Acoustics (AFA) Ultrasonication and Preparing the Isolated Nucleic Acid for Sequencing Via Hi-C

Dissociation of formalin fixed paraffin embedded (FFPE) samples was performed on a Covaris® M220 Focused-ultrasonicator using the microTUBE adapter. FFPE tissue slices were suspended in a solution of 1× Tris-Buffered Saline (TBS) with 0.1% sodium dodecyl sulfate (SDS) and proteinase K at a final concentration of 60 ng/4 in a 130 μL screw-cap microTUBE (Covaris item #500339). The solution was vortexed to mix and incubated at 37° C. for 10 minutes, with a brief vortex at 5 minutes. The microTUBE was subjected to Adaptive Focused Acoustics (AFA) ultrasonication using the following settings: Time: 5 min; Duty Factor: 20%; Peak Incident: 75W; 200 cycles/burst; 18-20° C.

The solution along with the tissue sample was transferred to a plastic microtube and heated to 98° C. for 10 minutes to inactivate the proteinase K. The solution was returned to the micoTUBE, which was then subjected to AFA ultrasonication using the following settings: 10 min; Duty Factor: 15%; Peak Incident: 75W; 200 cycles/burst; 4-7° C.

To recover nucleic acid material, the solution was transferred to a microtube and centrifuged for 5 minutes at 5,000×g. The supernatant was transferred to a new tube and the nucleic acid yield quantified using QUBIT fluorometric quantitation.

A Hi-C library was prepared. First, the nucleic acid material was bound to SPRI beads and washed twice with 1×CRB (1×TBS+1 mM EDTA). Subsequent steps were performed on the bead-bound nucleic acids. The nucleic acid material was fragmented by treatment with DpnII restriction endonuclease for 1 hour at 37° C., followed by biotinylation with T4 polymerase in the presence of biotin-dATP. The reaction was stopped with 500 mM EDTA at pH 8. Proximity ligation of blunted nucleic acid fragments was performed using T4 ligase at 25° C. for 4 hours, followed by heat inactivation at 65° C.

5 μL of Proteinase K at 20 ng/mL was added to the 100 μL sample (approximately 1 ng/mL final concentration) and the solution was incubated at 65° C. for at least one hour. The bead-bound library was washed with 20% PEG-8000, 2.5M NaCl and eluted from the beads using 10 mM Tris, pH 8.0, 0.1 mM EDTA.

The resulting biotinylated, proximity-ligated library was bound to streptavidin beads, which were washed twice with 1×NTB (5 mM Tris-HCl, pH 8.0, 0.5 mM EDTA, 1 M NaCl) and resuspended in 2×NTB (10 mM Tris-HCl, pH 8.0, 1 mM EDTA, 2 M NaCl) and incubated with blocking solution. The beads were washed twice with 1×NTB+0.5% Tween 20 and then once with 1×NTB, and resuspended in deionized water.

Nextera tagmentation was used to sequence the library. Tagmentation was performed essentially according to manufacturing instructions. The library was then amplified using Best 3.0 Polymerase and Illumina index primers, purified on SPRI beads, and subjected to high-throughput sequencing.

### Example 2-Demonstration of Next-Generation Cytogenomics by Proximity Ligation Sequencing

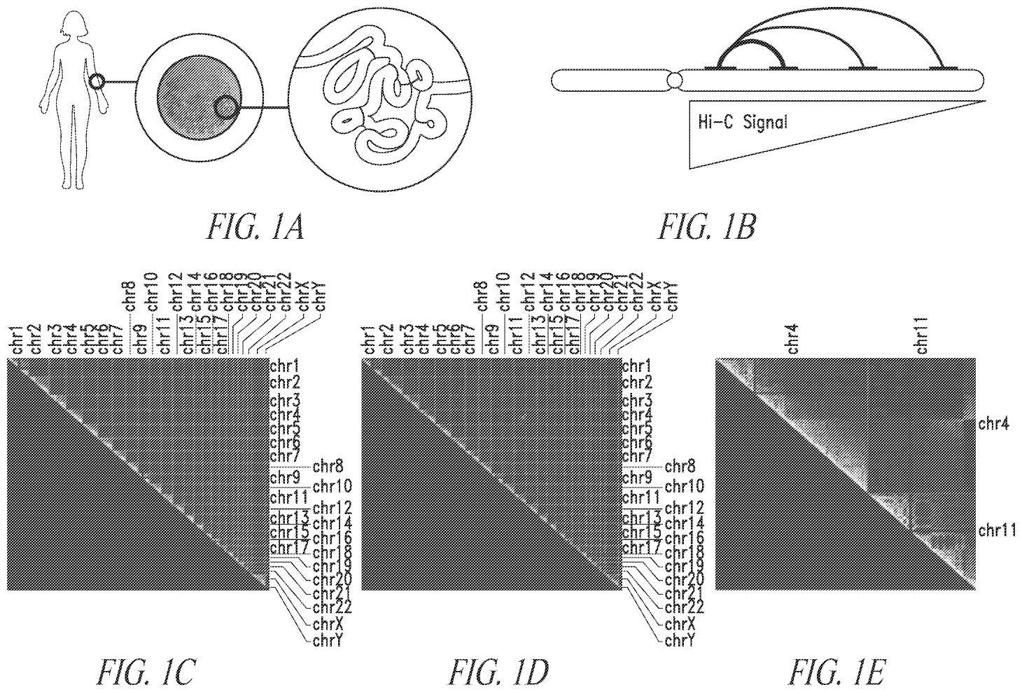

Hi-C has is a valuable tool in the scaffolding of genome sequences, ordering and orienting segments of DNA sequences into fully assembled chromosomes. The method begins by crosslinking chromatin in its native state within the intact nucleus (FIG. 1A). The crosslinks formed during formalin fixation are identical to those used in the Hi-C method making use of FFPE tissue possible. Cross-linked chromatin is fragmented; fragments are ligated to create chimeric sequences which can be sequenced using Illumina paired end chemistry. Sequencing these chimeric DNA molecules captures the signal of ultra-long-range chromatin interactions (such as promoter-enhancer interactions) but the overwhelming majority of the signal in proximity ligation sequencing reflects the linear distance between two sequences on a chromosome (FIG. 1B). This is easily observed when Hi-C is performed on the human genome and the mapping coordinates of the read pairs are plotted as a heatmap (FIG. 1C). In the case of a normal human genome, the pairs of sequences map along the diagonal, reflecting Hi-C read pairs mapping along the linear length of the chromosome. When Hi-C is performed on a sample containing a chromosomal aberration, this strict ordering of Hi-C read pairs along the diagonal is disrupted relative to the human reference genome. This is visualized in the case of a cancer cell line which exhibits a translocation between chromosome 4 and 11 (MV 4; 11, FIGS. 1D and 1E).

Illuminating chromosome aberrations in solid tumors: Chromosome aberrations in solid tumor biology have been historically difficult to determine. Karyotyping method are extremely difficult and often time impossible to apply to most solid tumors. Whole Genome Sequencing (WGS) surveys are also have limited practical value in detecting chromosome aberrations for several reasons. (1) WGS requires high coverage (30-60×) to detect aberrations with high confidence because there must be substantial coverage at the junction of the rearrangement. (2) Short read sequencing is insufficient to span the length of repetitive regions of the genome which frequently mediate rearrangements making identification of the rearrangement impossible. (3) Long read WGS which can often times span repetitive regions of the genome can successfully overcome the mapping limitations and identify breakpoints, but requires high molecular weight DNA which is difficult to extract and impossible to recover for FFPE tissue. Hi-C methods can surmount all three of these limitations, requiring only low-pass sequencing (1-5×), identifying breakpoints in repetitive regions of the genome by sequencing hundreds of reads that are proximal to repetitive sequence breakpoints, and is compatible with FFPE tissue.

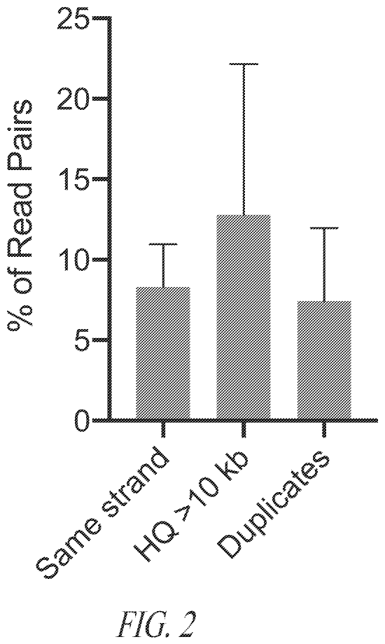

Open source library evaluation using HiC QC: To assist in evaluating library quality, criteria was established that define the performance of libraries from a small sample of reads from an FFPE Hi-C library generated using the method described in Example 1. Between 0.5-1M read pairs of sequence from the Hi-C library were used to judge library quality with the open source analytic tool, HiC_QC. Among the key parameters evaluated were: Same strand high quality read pairs: This was indicative that the read was the result of a proximity ligation event which changes the orientation of the sequences relative to each other. Doubling this value gave an estimate of the total percentage of Hi-C junctions present in the library. (5% minimum value was found acceptable). Fraction of high quality read pairs >10 kb apart: Hi-C library success is dependent on the fraction of reads that contain long-range contact information. This stat measured the percentage of high quality read pairs that map >10 kb apart in the reference genome. (2.5% minimum was found to be an acceptable value). Duplicate Reads: This measured the rate of PCR duplicate fragments present in the library and fits a saturation model to extrapolate the duplication rate at 100M read pairs. This is a critical measure of the complexity of a library. (40% maximum was found to be an acceptable value). Using these metrics, the FFPE Hi-C methods provided throughout this disclosure were found to be sufficient to meet the requirements for the KBS application (see FIG. 2).

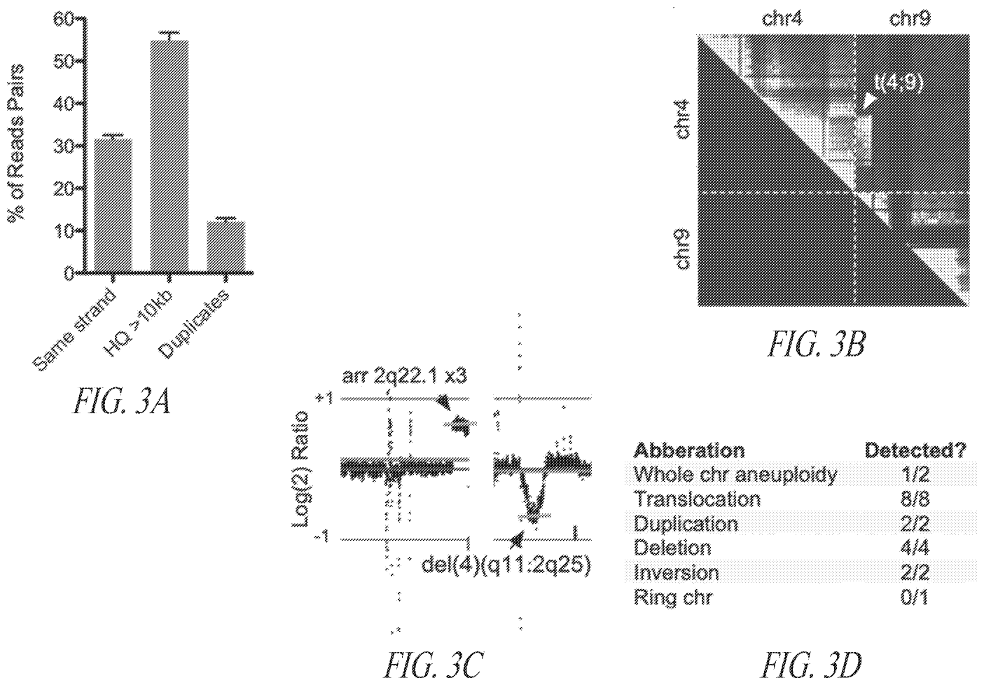

Hi-C libraries from clinical samples: To determine if Hi-C on clinical samples can meet the quality threshold necessary for cytogenomic testing, “off-the-shelf” academic software was utilized to identify copy number variants with HiNT and using hic_breakfinder to identify chromosome aberration breakpoints. Relying on previously well-characterized samples as a gold-standard, Hi-C was demonstrated to yield 2 false negative calls in 19 known aberrations (FIG. 3A-3D). Importantly the false negatives were low abundance (˜20%) aberrations and included an aberration for which hic_breakfinder is not currently optimized to detect (ring chromosomes). These values meet standards set for most cytogenomics tests with existing software and no optimization, albeit with a small sample size. Advancements in variant detection discussed below may further reduce false positive and negative rates reciprocally increasing the sensitivity and specificity of KBS.

Design and Methods

Design: A benchmarking study using the extensive experience Intermountain Precision Genomics and Phase Genomics will be conducted to evaluate the application of proximity ligation to cytogenomic testing. The benchmarking study will test the applicability of Hi-C proximity ligation sequencing to a cohort of triple negative breast cancer tumors, a class of cancer that has few actionable biomarkers. The triple negative breast cancer (TNBC) samples will be obtained through the Intermountain Biorepository. The study will have two related aims. First, it will be determined if the broad range of tissue sample collection methods used within a clinical cohort are sufficiently well-preserved to yield useful chromosome structure information. 200 Hi-C libraries will be generated from Intermountain Biorepository samples using the methods described in Example 1 and said Hi-C libraries will be sequenced by Intermountain Precision Genomics. Resulting data will be analyzed using the HiC_QC software described in this example using the criteria described therein to determine sufficiency. The second phase of the study will be to use the Hi-C sequencing data to determine the range of chromosome aberrations present in the TNBC samples. In the preliminary data section of this example, we describe results from ‘off’-the-shelf software solutions were described. Samples will be analyzed using Phase Genomics, Inc. proprietary Artificial Intelligence platform to define the classes and breakpoints of aberrations observed in TNBC. Within the scope of this limited study, outcomes will be associated with classes of aberrations observed.

Part 1: Benchmark the Performance of KBS on ‘Real-World’ FFPE Samples.

Methods: Sample selection criteria will be TNBC surgical resection samples identified from the Intermountain Biorepository for individuals who are no longer living and will be de-identified. We will work with Intermountain Biorepository to assure the appropriate IRB-approved exemptions for whole genome sequencing are in place if applicable.

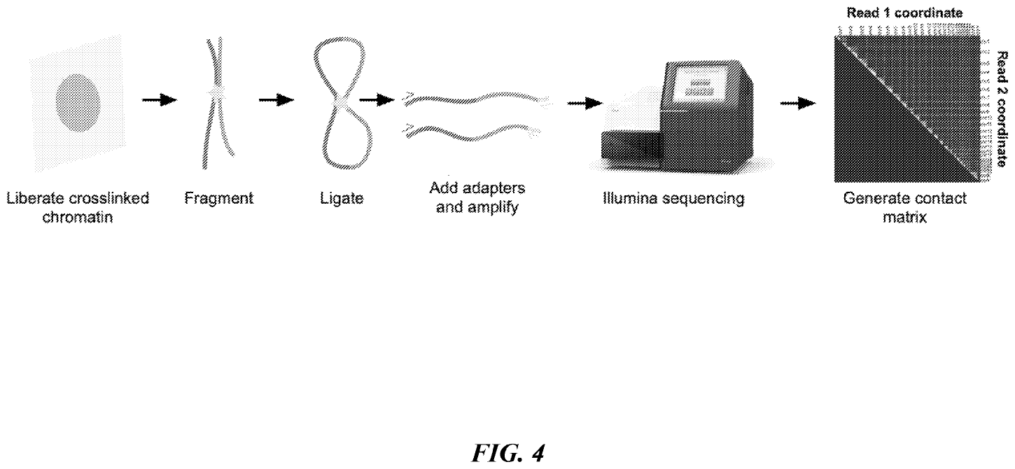

All FFPE samples are cross-linked in their native state creating covalent bonds between chromatin that are in close proximity within the nucleus (FIG. 4). The chromatin from two 5 μm FFPE curls will be liberated using focused acoustic energy (AFA ultrasonication) without shearing and prepared for Hi-C. The liberated chromatin will be processed for DNA fragmentation by restriction enzyme digestion. Overhanging sequences created by restriction digest will be filled in with biotinylated nucleotides and ligated together forming chimeric DNA molecules. Streptavidin beads will be used to purify sequences containing ligation junctions and will be used as a template to create an Illumina-compatible sequencing library. Based on preliminary data, as little as 30M read pairs is estimated to suffice for structural variant (SV) calling purposes. However, an increased amount of sequencing is anticipated to be required to detect complex rearrangements in a mixed population of normal and cancer cells. To empirically determine these thresholds, sequencing will be done to a depth of 10× whole genome coverage and downsampling the sequencing data to understand coverage requirements will be performed.

Interpretation of Results: Sequencing data will be analyzed using the open source analysis software HiC_QC. As described in the Preliminary Data section, HiC_QC evaluates a variety of library statistics which were identified as informative of library quality. As highlighted above, the percent of read pairs mapping to the same strand, long range (>10 kbp) interactions, and PCR/optical duplicates will be used, among other measures to determine how effective the described methods for chromatin extraction from FFPE samples are for evaluating structural variation and chromosome aberrations.

Part 2: Define the Capabilities of KBS to Detect Chromosome Aberrations in ‘Real-World’ FFPE Tissue Sections.

Methods: A software pipeline is being developed that (a) maps Hi-C data to a human reference genome to generate a contact frequency matrix; (b) analyzes said contact frequency matrix using a trained convolutional neural net (CNN), as well as a background model for healthy genome structure, to identify the location and type of possible SVs including copy number variants (CNVs) in the sample), and (c) cross-references detected variants with known clinical information to provide a report similar to those generated by traditional cytogenetic methods. This pipeline will be integrated into Phase Genomics' existing cloud-based platform to enable uploading and analyzing samples via the Phase Genomic website.

CNN Model Design: Based on preliminary results, two common CNN architectures were found, resnet-50 and RetinaNet that provide a suitable starting point for the detection of structural variants in Hi-C matrixes. Using a small simulated Hi-C dataset in a modified resnet-50 network, 96.5% accuracy was achieved for detecting the presence of unbalanced translocations in a sample, with a loss of 3.29%. The bounding box of such translocations was identified with an accuracy of 59.5% and a loss of 3.58%. Testing the same data in RetinaNet, an average precision in excess of 95% was achieved for detecting the location simulated events over 1 Mbp, a significant improvement over the more generic resnet-50 network. These results demonstrate that performance at least comparable to karyotyping is achievable with this approach, despite only using a small amount of simulated data and a relatively stock CNN. With additional training data, customization of the CNN model (including testing other network approaches such as that illustrated by yolo-v3), and identification of optimal hyperparameters, we expect to be able to develop a model with performance characteristics at least equal to, if not in excess of, the best results karyotype-based methods can achieve. Due to the nature of identifying events with CNNs, a variant-class label and confidence score for each call made by the CNN will also be produced and can be used to classify events and filter out low-confidence events to improve sensitivity and specificity. Using this computational pipeline we will infer the structure of genome rearrangements present within the 200 samples sequenced in Aim1 of the proposal.

Interpretation of Results: Based on the limited previous studies, we anticipate that we will observe at least 6 recurrent balanced translocations within the cohort obtained from Intermountain Biorepository. The very high rate of structural variants observed in previous studies of breast adenocarcinoma (>300 per tumor) using WGS suggests that we will observe a large number of other, unbalanced rearrangements. It is likely that a significant fraction of these events are the result of chaotic chromothripsis events and do not reflect ‘simple’ deletions, insertions, inversions, or translocations. Unlike WGS, the long-range sequence information that is recovered by Hi-C is able deconvolve these complex events and will yield a high proportion of phased events. This will result if in a more complete karyotype of events than can be resolved by existing technologies for FFPE tissue. The resulting catalog of chromosome aberrations will be used in exploratory data analysis to identify any potential stratification in patient outcomes.

### Example 3 Comparison of Methods for Generating Hi-C Libraries from Preserved Tissues Samples

The objective of this example will be to determine and compare the quality of Hi-C libraries generated using Hi-C on nucleic acid isolated from formalin-fixed, paraffin-embedded (FFPE) tissue samples using either a chemical-based FFPE nucleic acid extraction procedure or an Adaptive Focused Acoustic (AFA)-based FFPE nucleic acid extraction procedure. The AFA-based FFPE extraction procedure used in this example will not entail shearing the nucleic acid prior to performing Hi-C.

Hi-C library generation using a chemical-based FFPE nucleic acid extraction procedure will be performed as described in WO2017197300, which is incorporated herein by reference. Hi-C library generation using an AFA-based FFPE nucleic acid extraction procedure will be performed using the method described in Example 1 presented herein.

Following Hi-C library generation using nucleic acid extracted from FFPE using either of the FFPE nucleic acid extraction methods described in this example, the Hi-C libraries will be sequenced using Illumina NGS sequencing methods as described in Example 1 above.

In order to assess Hi-C library quality for each of the FFPE extraction methods, two critical features (i.e., (1) library complexity and (2) long range information) will be assessed. Library complexity will be directly measured by determining the percentage of reads from the NGS sequencing of each Hi-C library that are unique, or conversely the number of duplicate reads. Duplicate reads arise typically as a result of PCR amplification with less complex libraries leading to a higher rate of duplicates. Duplicate reads will be measured during the library quality control process using SAMBlaster, an open-source utility that is widely used by the next-generation sequencing community. The more complex a library is, the more potentially useful information is present.

Long range information can refer to the distance along the length of the chromosome between which Hi-C read pairs map. Hi-C read pairs spanning all distances can be useful, but more distant contacts (i.e. greater than 10 kbp) are less common and shorter range contacts due to the dynamics of chromosome conformation. The presence of long range Hi-C read pairs can help to improve ability Hi-C computational analysis to determine the structure of chromosomes and will be ascertained for the Hi-C libraries generated from nucleic acid isolated from either of the FFPE extraction methods described in this example. Reductions in long range information in a Hi-C library can typically be due to low sample quality or problem in library preparation methodology.

### Example 4-Demonstrating the Utility of Adaptive Focused Acoustics (AFA) Ultrasonication for Preparing and Analyzing Hi-C Libraries from Clinical FFPE Samples

The objective of this example was to demonstrate the utility of AFA ultrasonication for extracting nucleic acid from clinical formalin-fixed, paraffin-embedded (FFPE) breast and ovary tissue samples, generating Hi-C libraries therefrom and analyzing the Hi-C libraries to identify the presence of non-reciprocal translocations. The AFA-based FFPE extraction procedure used in this example was similar to the AFA ultrasonication nucleic acid extraction outlined in Example 1, but differs in that it employs an additional dissociating step. Moreover, the presence of non-reciprocal translocations in the Hi-C libraries generated from the breast and ovary clinical samples used in this method was determined using the analytical methods described in Example 2 (e.g., Part 2-CNN model) on next-generation sequencing data (i.e., Illumina sequencing) obtained from the Hi-C libraries as described in Example 1.

Extraction of Nucleic Acid from FFPE Breast and Ovary Tumor Samples Using Adaptive Focused Acoustics (AFA) Ultrasonication

Dissociation of each of the formalin fixed paraffin embedded (FFPE) breast and ovary tumor samples was performed on a Covaris® M220 Focused-ultrasonicator using the microTUBE AFA Fiber Pre-silt Snap-Cap 6×16 mm tubes as follows. FFPE curls from each tumor sample were individually suspended in 100 microliters of Lysis Buffer 2 (10 mM Tris, 150 mM sodium chloride, 0.1% sodium dodecyl sulfate (SDS), pH 7.5) to which 0.3 microliters of 20 mg/ml proteinase K was added. The solution was mixed by vortexing and incubated at 37° C. on a heat block for 5 minutes. The microTUBE was then moved to the Covaris® M220 AFA ultrasonicator and subjected to Adaptive Focused Acoustics (AFA) ultrasonication using the following settings: Time: 5 min; Duty Factor: 20%; Peak Incident: 75W; 200 cycles/burst; 18-20° C.

For both the breast and ovary samples, the supernatant (i.e., supernatant 1) was transferred to 0.2 ml PCR tube and stored at 4° C., while leaving the solids behind in the Covaris microTUBE. One hundred (100) microliters of Lysis Buffer 2 (10 mM Tris, 150 mM sodium chloride, 0.1% SDS, pH 7.5) and 0.3 microliters of 20 mg/ml proteinase K was added to the solids remaining in the microTUBE and incubated at 37° C. on a heat block for 5 minutes. The solution was then subjected to AFA ultrasonication using the following settings: 5 min; Duty Factor: 20%; Peak Incident: 75W; 200 cycles/burst; 18-20° C.

For both the breast and ovary samples, the supernatant (i.e., supernatant 2) was transferred to 0.2 ml PCR tube and stored at 4° C., while leaving the solids behind in the Covaris microTUBE. Both supernatant 1 and supernatant 2 were then incubated in their respective 0.2 ml PCR tubes at 98° C. for 10 minutes to inactivate any remaining proteinase K and then stored at 4° C. until the AFA ultrasonicator cooled to 4° C. Each of supernatant 1 and 2 were then transferred from the PCR tubes to fresh Covaris microTUBE AFA Fiber Pre-Slit Snap-Cap 6×16 mm tubes. Each microTUBE containing either supernatant 1 or 2 was then subjected to AFA ultrasonication using the following settings: 10 min; Duty Factor: 15%; Peak Incident: 75W; 200 cycles/burst; 4-7° C. The supernatants were then combined in a 1.5 ml microcentrifuge tube.

For both the breast and ovary samples, to recover the nucleic acid material, an equal volume of Solid Phase Reversible Immboilization (SPRI) beads were added to the combined supernatants. After allowing chromatin to bind to the SPRI beads for 10 minutes at room temperature, the beads were placed on magnetic rack, permitting the removal of supernatant. The beads off the magnetic rack were washed once with 200 microliters of 10 mM Tris, 150 mM sodium chloride, 0.1 mM ethylenediaminetetraacetic acid, pH 7.5. Following wash, the beads were once again placed on the magnetic rack and the wash solution removed.

For both the breast and ovary samples, a Hi-C library was prepared from the bead-bound nucleic acid material. The nucleic acid material was fragmented by treatment with DpnII restriction endonuclease for 1 hour at 37° C., followed by end repair with T4 polymerase in the presence of biotin-dATP. The reaction was stopped with 20 mM EDTA at pH 8. Proximity ligation of blunted nucleic acid fragments was performed using T4 ligase at 25° C. for 4 hours, followed by heat inactivation at 65° C.

5 μL of Proteinase K at 20 mg/mL was added to the 100 μL sample (approximately 1 ng/mL final concentration) and the solution was incubated at 65° C. for at least one hour. The library bound to beads was washed with 20% PEG-8000, 2.5M NaCl, washed twice with 80% ethanol, and, following air drying of the beads, eluted from the beads using 10 mM Tris, pH 8.0, 0.1 mM EDTA.

For both the breast and ovary samples, the resulting biotinylated, proximity-ligated library was bound to streptavidin beads, which were washed twice with 1×NTB (5 mM Tris-HCl, pH 8.0, 0.5 mM EDTA, 1 M NaCl) and resuspended in 2×NTB (10 mM Tris-HCl, pH 8.0, 1 mM EDTA, 2 M NaCl) and incubated with blocking solution. The beads were washed twice with 1×NTB+0.5% Tween 20 and then once with 1×NTB, and resuspended in deionized water.

For both the breast and ovary samples, Nextera tagmentation was used to generate an Illumina-compatible sequencing library. Tagmention was performed essentially according to manufacturing instructions. The library derived from each of the breast and ovary samples was then amplified using a mixture of high-fidelity polymerase chain reaction enzymes, Bst 3.0 Polymerase and Illumina index primers, purified on SPRI beads, and subjected to high-throughput sequencing.

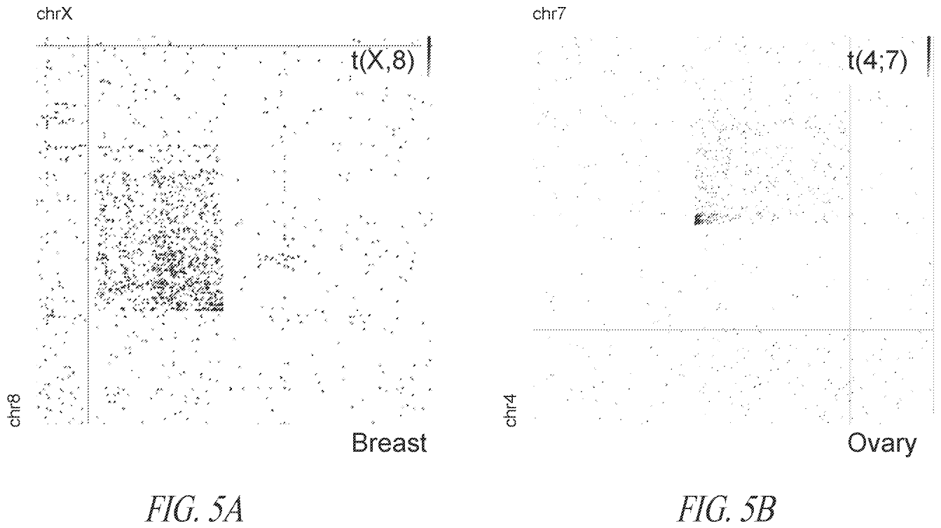

The sequencing data obtained from the libraries generated from both the breast and ovary samples were then analyzed for the presence of chromosomal rearrangements using the analytical methods described in the Examples provided herein. Specifically, paired-end Hi-C reads were aligned to a human reference genome (e.g., HG19, HG38, a representative genome from a human pangenome reference set of an appropriate background, or a de novo assembly of healthy tissue from the individual from which the sample was obtained) using an alignment method (e.g., Burrows-Wheeler alignment, local alignment, gapped alignment, paired-end alignment). A matrix was constructed from these alignments by a series of steps. First, a resolution was chosen or determined empirically from the data. Second, the genome was binned at the chosen resolution. Third, individual aligned read pairs were examined to determine which genome bins (x, y) corresponded to each aligned read pair and counted in the matrix at the corresponding (x, y) coordinates. Before, during, or after this counting process, aligned read pairs which had insufficient quality, which were secondary or non-primary, which may have originated as side effects of biochemical procedures such as duplication by polymerase chain reaction (PCR) processes, or which were otherwise undesirable were excluded from the counting. The matrix now contained “linkage counts” expressing the number of times a chromatin conformation read pair was observed linking all pairs of genome bins. Fourth, the matrix was normalized to account for sources of bias such as choice of restriction enzyme(s) used during sample preparation, the read depth observed in a given genome bin, size or sequence variation within the genome bins, biological factors known a priori about the genome (such as the expected number and type of sex chromosomes in the genome), or other possible sources of noise. The matrix now contained “linkage densities” which expressed how often a randomly formed chromatin conformation read pair would join each pair of genome bins. Fifth, the matrix was visualized in a 2-D graph or heatmap. Aberrations in the expected statistical properties of linkage densities were often visible to the eye in these figures. For example, as shown in FIGS. 5A and 5B, translocations between chromosomes were visible as blocks of increased linkage density with clear edges and a distinct corner. These blocks resulted from the fact that, for the sequences in those regions, the reference genome had those sequences on a different chromosome than they were on in the sample, and because chromatin conformation read pairs form at a rate of an order of magnitude or greater more often for sequences on the same molecule, the chromatin conformation reads for translocated sequences express linkage densities far greater than one would expect in the reference genome alone.

Results/Conclusions

As shown in FIGS. 5A and 5B, libraries generated using above described methods from a single section of FFPE breast (FIG. 5A) or ovary (FIG. 5B) tumor sample was sufficient to identify non-reciprocal translocations between chromosomes X and 8 in the breast tumor sample (FIG. 5A) and chromosomes 4 and 7 in the ovary tumor sample (FIG. 5B).

### Numbered Embodiments of the Disclosure

Other subject matter contemplated by the present disclosure is set out in the following numbered embodiments:

1. A method, comprising:
providing a tissue sample in a solution in a vessel, the tissue sample comprising nucleic acid material;
dissociating the tissue sample by exposing the tissue sample and the solution in the vessel to focused acoustic energy to release the nucleic acid material from the tissue sample;
recovering the nucleic acid material; and
performing chromosome conformation capture analysis on the nucleic acid material.

2. The method of embodiment 1, wherein the solution is a non-solvent solution.

3. The method of embodiment 1 or 2, wherein the tissue sample is a preserved tissue sample.

4. The method of any one of the above embodiments, wherein the tissue sample is a cross-linked tissue sample.

5. The method of any one of the above embodiments, wherein the tissue sample is a formalin fixed paraffin-embedded (FFPE) sample.

6. The method of embodiment 5, wherein the disassociating step comprises exposing the FFPE sample to focused acoustic energy for a time sufficient to disassociate enough paraffin from the FFPE sample to allow recovery of the nucleic acid material from the tissue sample.

7. The method of embodiment 5 or 6, wherein the disassociating step comprises disassociating more than 90% of paraffin attached to the FFPE sample.

8. The method of any one of embodiments 5-7, wherein the disassociating step comprises disassociating more than 98% of paraffin attached to the FFPE sample.

9. The method of any one of the above embodiments, wherein the disassociating step comprises rehydrating the tissue sample while exposing the tissue sample to focused acoustic energy.

10. The method of any one of the above embodiments, wherein the disassociating step comprises maintaining a temperature of the solution at about 5° C. to about 60° C. or about 18° C. to about 20° C.

11. The method of any one of the above embodiments, wherein the tissue sample has a thickness of 5 to 25 microns and a length of less than 25 mm.

12. The method of any one of the above embodiments, wherein the dissociating step comprises adding a protease to the solution and the tissue sample in the vessel prior to exposing the tissue sample to focused acoustic energy.

13. The method of embodiment 12, comprising inactivating the protease.

14. The method of embodiment 13, wherein the inactivating the protease comprises heating the vessel to about 98° C.

15. The method of any one of the above embodiments, comprising maintaining the tissue sample in the vessel at below 50° C. until heating with sample to 90-100° C.

16. The method of any one of the above embodiments, wherein the focused acoustic energy has a duty factor of between 10% and 30%.

17. The method of embodiment 16, wherein the focused acoustic energy has a duty factor of about 15% or about 20%.

18. The method of any one of the above embodiments, wherein the focused acoustic energy has a peak intensity power of between 60W and 90W.

19. The method of embodiment 18, wherein the focused acoustic energy has a peak intensity power of about 75W.

20. The method of any one of the above embodiments, further comprising performing a second dissociating step comprising exposing the tissue sample and the solution in the vessel to focused acoustic energy to release additional nucleic acid material from the tissue sample while maintaining the vessel at about 4° C. to about 7° C.

21. The method of embodiment 20, wherein the focused acoustic energy has a duty factor of between 10% and 30%.

22. The method of embodiment 20, wherein the focused acoustic energy has a duty factor of about 15% or about 20%.

23. The method of any one of embodiments 20-22, wherein the focused acoustic energy has a peak intensity power of between 60W and 90W.

24. The method of embodiment 23, wherein the focused acoustic energy has a peak intensity power of about 75W.

25. The method of any one of embodiments 1-19, further comprising isolating supernatant following the dissociating step in a vessel, adding additional solution to the vessel comprising the tissue sample and performing a second dissociating step on the tissue sample comprising exposing the tissue sample and the additional solution in the vessel to focused acoustic energy to release additional nucleic acid material from the tissue sample while maintaining the vessel at about 5° C. to about 60° C. or about 18° C. to about 20° C.

26. The method of embodiment 25, wherein the focused acoustic energy has a duty factor of between 10% and 30%.

27. The method of embodiment 20, wherein the focused acoustic energy has a duty factor of about 15% or about 20%.

28. The method of any one of embodiments 25-27, wherein the focused acoustic energy has a peak intensity power of between 60W and 90W.

29. The method of embodiment 28, wherein the focused acoustic energy has a peak intensity power of about 75W.

30. The method of any one of embodiments 25-29, further comprising isolating supernatant following the second dissociating step in a vessel, performing a third dissociating step on both the supernatant isolated following the second dissociating step and the supernatant isolated prior to the second dissociating step by exposing each of the supernatants to focused acoustic energy while maintaining the temperature of the vessels comprising the supernatants at about 4° C. to about 7° C. and combining the supernatants.

31. The method of embodiment 30, wherein the focused acoustic energy has a duty factor of between 10% and 30%.

32. The method of embodiment 30, wherein the focused acoustic energy has a duty factor of about 15% or about 20%.

33. The method of any one of embodiments 30-32, wherein the focused acoustic energy has a peak intensity power of between 60W and 90W.

34. The method of embodiment 33, wherein the focused acoustic energy has a peak intensity power of about 75W.

35. The method of any one of the above embodiments, wherein the dissociating step comprises exposing the tissue sample to focused acoustic energy at an intensity suitable to avoid shearing the nucleic acid material.

36. The method of any one of the above embodiments, wherein a majority of the fragments of nucleic acid material after exposing the tissue sample to focused acoustic energy have a size of 1000 bp or greater.

37. The method of any one of the above embodiments, wherein the dissociating step preserves formaldehyde crosslinks in the tissue sample.

38. The method of any one of the above embodiments, wherein the focused acoustic energy has a frequency of between about 100 kilohertz and about 100 megahertz; the focused acoustic energy has a focal zone with a width of less than about 2 centimeters; and/or the focused acoustic energy originates from an acoustic energy source spaced from and exterior to the vessel, wherein at least a portion of the acoustic energy propagates exterior to the vessel.

39. The method of any one of the above embodiments, wherein the recovering step comprises centrifuging the tissue sample, thereby separating a supernatant solution containing nucleic acid material dissociated from insoluble contaminants.

40. The method of any one of embodiments 1-38, wherein the recovering step comprises purifying nucleic acid material by solid phase reversible immobilization.

41. The method of any one of the above embodiments, wherein performing chromosome conformation capture analysis on the nucleic acid material comprises: proximity ligating the nucleic acid material to form a library of proximity-ligated polynucleotides and identifying paired polynucleotide sequences in the library of proximity-ligated polynucleotides.

42. The method of any one of embodiments 1-40, wherein performing chromosome conformation capture analysis on the nucleic acid material comprises: fragmenting the nucleic acid material, proximity ligating the nucleic acid material to form a library of proximity-ligated polynucleotides, and identifying paired polynucleotide sequences in the library of proximity-ligated polynucleotides.

43. The method of embodiment 41 or embodiment 42, wherein the identifying step comprising sequencing the proximity ligations.

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method, comprising:
a) providing a tissue sample in a solution in a vessel, the tissue sample comprising nucleic acid material;
b) dissociating the tissue sample by exposing the tissue sample and the solution in the vessel to focused acoustic energy to release the nucleic acid material from the tissue sample;
c) recovering the nucleic acid material; and
d) performing chromosome conformation capture analysis on the nucleic acid material.

2. The method of claim 1, wherein the solution is a non-solvent solution.

3. The method of claim 1, wherein the tissue sample is a preserved tissue sample.

4. The method of claim 1, wherein the tissue sample is a cross-linked tissue sample.

5. The method of claim 1, wherein the tissue sample is a formalin fixed paraffin-embedded (FFPE) sample.

6. The method of claim 5, wherein the disassociating step comprises exposing the FFPE sample to focused acoustic energy for a time sufficient to disassociate enough paraffin from the FFPE sample to allow recovery of the nucleic acid material from the tissue sample.

7. The method of claim 5, wherein the disassociating step comprises disassociating more than 90% of paraffin attached to the FFPE sample.

8. The method of claim 5, wherein the disassociating step comprises disassociating more than 98% of paraffin attached to the FFPE sample.

9. The method of claim 1, wherein the disassociating step comprises rehydrating the tissue sample while exposing the tissue sample to focused acoustic energy.

10. The method of claim 1, wherein the disassociating step comprises maintaining a temperature of the solution at about 5° C. to about 60° C. or about 18° C. to about 20° C.

11. The method of claim 1, wherein the tissue sample has a thickness of 5 to 25 microns and a length of less than 25 mm.

12. The method of claim 1, wherein the dissociating step comprises adding a protease to the solution and the tissue sample in the vessel prior to exposing the tissue sample to focused acoustic energy.

13. The method of claim 12, comprising inactivating the protease.

14. The method of claim 13, wherein the inactivating the protease comprises heating the vessel to about 98° C.

15. The method of claim 1, comprising maintaining the tissue sample in the vessel at below 50° C. until heating with sample to 90-100° C.

16. The method of claim 1, wherein the focused acoustic energy has a duty factor of between 10% and 30%.

17. The method of claim 16, wherein the focused acoustic energy has a duty factor of about 15% or about 20%.

18. The method of claim 1, wherein the focused acoustic energy has a peak intensity power of between 60W and 90W.

19. The method of claim 18, wherein the focused acoustic energy has a peak intensity power of about 75W.

20. The method of claim 1, further comprising performing a second dissociating step comprising exposing the tissue sample and the solution in the vessel to focused acoustic energy to release additional nucleic acid material from the tissue sample while maintaining the vessel at about 4° C. to about 7° C.

21. The method of claim 20, wherein the focused acoustic energy has a duty factor of between 10% and 30%.

22. The method of claim 20, wherein the focused acoustic energy has a duty factor of about 15% or about 20%.

23. The method of claim 20, wherein the focused acoustic energy has a peak intensity power of between 60W and 90W.

24. The method of claim 23, wherein the focused acoustic energy has a peak intensity power of about 75W.

25. The method of claim 1, further comprising isolating supernatant following the dissociating step in a vessel, adding additional solution to the vessel comprising the tissue sample and performing a second dissociating step on the tissue sample comprising exposing the tissue sample and the additional solution in the vessel to focused acoustic energy to release additional nucleic acid material from the tissue sample while maintaining the vessel at about 5° C. to about 60° C. or about 18° C. to about 20° C.

26. The method of claim 25, wherein the focused acoustic energy has a duty factor of between 10% and 30%.

27. The method of claim 20, wherein the focused acoustic energy has a duty factor of about 15% or about 20%.

28. The method of claim 25, wherein the focused acoustic energy has a peak intensity power of between 60W and 90W.

29. The method of claim 28, wherein the focused acoustic energy has a peak intensity power of about 75W.

30. The method of claim 25, further comprising isolating supernatant following the second dissociating step in a vessel, performing a third dissociating step on both the supernatant isolated following the second dissociating step and the supernatant isolated prior to the second dissociating step by exposing each of the supernatants to focused acoustic energy while maintaining the temperature of the vessels comprising the supernatants at about 4° C. to about 7° C. and combining the supernatants.

31. The method of claim 30, wherein the focused acoustic energy has a duty factor of between 10% and 30%.

32. The method of claim 30, wherein the focused acoustic energy has a duty factor of about 15% or about 20%.

33. The method of claim 30, wherein the focused acoustic energy has a peak intensity power of between 60W and 90W.

34. The method of claim 33, wherein the focused acoustic energy has a peak intensity power of about 75W.

35. The method of claim 1, wherein the dissociating step comprises exposing the tissue sample to focused acoustic energy at an intensity suitable to avoid shearing the nucleic acid material.

36. The method of claim 1, wherein a majority of the fragments of nucleic acid material after exposing the tissue sample to focused acoustic energy have a size of 1000 bp or greater.

37. The method of claim 1, wherein the dissociating step preserves formaldehyde crosslinks in the tissue sample.

38. The method of claim 1, wherein the focused acoustic energy has a frequency of between about 100 kilohertz and about 100 megahertz; the focused acoustic energy has a focal zone with a width of less than about 2 centimeters; and/or the focused acoustic energy originates from an acoustic energy source spaced from and exterior to the vessel, wherein at least a portion of the acoustic energy propagates exterior to the vessel.

39. The method of claim 1, wherein the recovering step comprises centrifuging the tissue sample, thereby separating a supernatant solution containing nucleic acid material dissociated from insoluble contaminants.

40. The method of claim 1, wherein the recovering step comprises purifying nucleic acid material by solid phase reversible immobilization.

41. The method of claim 1, wherein performing chromosome conformation capture analysis on the nucleic acid material comprises: proximity ligating the nucleic acid material to form a library of proximity-ligated polynucleotides and identifying paired polynucleotide sequences in the library of proximity-ligated polynucleotides.

42. The method of claim 1, wherein performing chromosome conformation capture analysis on the nucleic acid material comprises: fragmenting the nucleic acid material, proximity ligating the nucleic acid material to form a library of proximity-ligated polynucleotides, and identifying paired polynucleotide sequences in the library of proximity-ligated polynucleotides.

43. The method of claim 41, wherein the identifying step comprising sequencing the proximity ligations.

44. The method of claim 42, wherein the identifying step comprising sequencing the proximity ligations.
